(19) United States
(12) Patent Application Publication
(10) Pub. No.: US 2025/0265798 A1
(43) Pub. Date: Aug. 21, 2025

(54) THREE-DIMENSIONAL MODELING OF INTERPROXIMAL SPACES

(71) Applicant: Align Technology, Inc., San Jose, CA (US)

(72) Inventors: Ilya ARKHIPOVSKIY, Moscow (RU); Evgeny LIPOVETSKY, Rishon LeZion (IL); Gal PELEG, Kiryat-Ono (IL); Vitaly SURAZHSKY, Yokneam Illit (IL)

(21) Appl. No.: 19/054,772

(22) Filed: Feb. 14, 2025

Related U.S. Application Data
(60) Provisional application No. 63/554,120, filed on Feb. 15, 2024.

(51) Int. Cl. G06T 19/20; A61B 1/00; A61B 1/24; A61C 9/00; A61C 13/34; G06T 7/33; G06T 7/521; G06T 7/55

(52) U.S. Cl. CPC G06T 19/20; A61B 1/00172; A61B 1/24; A61C 9/006; A61C 13/34; G06T 7/344; G06T 7/521; G06T 7/55; G06T 2207/10028; G06T 2207/10048; G06T 2207/10064; G06T 2207/30036; G06T 2210/41; G06T 2210/56

(57) ABSTRACT

Methods and apparatuses that may improve the accuracy of three-dimensional models from interproximal regions of intraoral scan data using non-structured light illumination images (e.g., white light, near-infrared light, fluorescent light, etc.). These methods and apparatuses may correct irregularities (e.g., holes, gaps, etc.) in the 3D digital model of the subject's teeth and may enhance treatment planning and the accuracy and effectiveness of dental appliances.

### CLAIM OF PRIORITY

This application claims priority to U.S. Provisional Patent Application No. 63/554,120, filed on Feb. 15, 2024 and titled “THREE-DIMENSIONAL MODELING OF INTERPROXIMAL SPACES,” herein incorporated by reference in its entirety.

### BACKGROUND

Intraoral scanners are capable of generating detailed three-dimensional models of a subject's dentition, and may scan the subject's teeth in real time, as the scanning cameras are moved relative to the subject's teeth. In some cases, the three-dimensional model may be generated using a patterned (or non-uniform) illumination technique, such as structured light, to rapidly generate a three-dimensional (3D) digital model. Although such scanners may be surprisingly accurate even when rapidly scanned over the subject's teeth, the resolution of such 3D models may be lower than desired. This may lead to a lack of some fine details, even when scanning with multiple cameras simultaneously.

It would be beneficial to provide methods and apparatuses that may be used with or integrated into intraoral scanning to improve the resulting scanned digital models of the teeth. In particular, it would be useful to improve scanning of interproximal regions, e.g., regions between teeth, for which fewer scanning points may be taken using structured light. Interproximal regions scanned using structured light to capture the 3D surface may have a limited resolution.

Described herein are methods and apparatuses that may improve the results of intraoral scanning and the analysis/interpretation of intraoral scans and resulting 3D models, particularly within interproximal regions of the subject's dentition.

### SUMMARY OF THE DISCLOSURE

Intraoral scanners may provide very rapid imaging and three-dimensional (3D) modeling of a subject's teeth, but may result in regions of fidelity scanning, particularly in recessed or partially obstructed regions. For example interproximal regions, e.g., the regions between adjacent teeth, may be challenging to scan and model. In some cases, the intraoral scanner may use patterned illumination (e.g., structured light, patterned confocal illumination, etc.) when performing an intraoral scan of the teeth, which involves employing patterned, non-uniform illumination for creating a map, and may generate a point cloud as part of the 3D modeling technique; interproximal regions may have fewer points in the point cloud. However, in some cases the intraoral scanner may use one or more additional imaging modes, especially un-patterned illumination (e.g., non-structured light illumination) modes such as “white light,” near-IR, fluorescent light, or other visible light modes. These additional imaging modes can be captured in between the structured light images and may be referred to generally as un-patterned illumination, non-structured light illumination, or nonstructured light imaging. Note that for convenience, the example herein typically refer to un-patterned illumination as non-structured light illumination, but may generally refer to any un-patterned illumination; similarly for convenience, patterned illumination is generally referred to as structured light illumination, but may refer to any patterned light illumination, including patterned confocal light illumination that may be used to generate a point cloud, and is not limited to just structured light examples.

As used herein, non-structured light (e.g., un-patterned) illumination is distinct from structured light (e.g., patterned) illumination, and, as mentioned above, may be referred to as uniform illumination or un-patterned illumination, since these modes of illumination do not project features (e.g., high contrast regions) that can be used for positioning. Un-patterned illumination, e.g., non-structured light illumination or uniform illumination, may include any appropriate wavelength or range of wavelengths, including color imaging, near-IR imaging and confocal imaging. Many intraoral scanners may use multiple different modalities (including alternating structured light imaging with non-structured light imaging). Such systems may face the technical challenge of accurately positioning the non-structured light illumination images relative to the 3D scan modality. As used herein, uniform illumination typically refers to illumination that is spread over the entire field of view, but may have regions of different intensity.

For example, described herein are methods comprising: identifying an interproximal region including a surface hole in a 3D model derived from a plurality of patterned illumination images (e.g., structured light, patterned confocal images, etc.) from an intraoral scan of a patient's teeth; identifying a set of un-patterned illumination (e.g., non-structured light illumination) images taken during the intraoral scan of the patient's teeth, including a region of the patient's teeth corresponding to the interproximal region including the surface hole in the 3D model; optionally modifying a camera position of a camera corresponding to each of the non-structured light illumination images of the set with an alignment transform derived from a comparison of features in a features map of the one 3D model relative to the camera with corresponding features from the un-patterned illumination image; and correcting the surface hole in the 3D model using one or more points generated from the modified camera positions and the un-patterned illumination images in combination with points from a point cloud derived from the patterned illumination images from which the 3D model was derived. In variations in which the same 2D image is used to provide both the patterned illumination image and the un-patterned illumination image (therefore having the identical and correct camera position for both), such as when only the un-patterned portion of the 2D image is used, the alignment step (e.g., modifying the camera position). Instead, the method and/or apparatus may include correcting the surface hole in the 3D model using one or more points generated from the un-patterned illumination images in combination with points from a point cloud derived from the patterned illumination images from which the 3D model was derived.

The un-patterned illumination images may comprise one or more of: white light images, near infrared (near-IR) images, and/or fluorescent images. Any of these methods may include removing any un-patterned illumination images from the set of un-patterned illumination images in which a camera angle between a camera taking the un-patterned illumination image and the region corresponding to the interproximal region including the surface hole is greater than a threshold and/or wherein the camera separated from the surface of the region corresponding to the interproximal region by a distance that is greater than a distance threshold value. The features map may correspond to a depth map. In some examples the features map corresponds to a height map.

In examples using the one or more points generated from the modified camera positions and the un-patterned illumination images in combination with points from a point cloud are used, this step may include using a radial basis function.

Correcting the surface hole in the 3D model using one or more points generated from the modified camera positions and the un-patterned illumination images may include identifying the one or more points from rays projected from the modified camera position to the region of the patient's teeth corresponding to the interproximal region including the surface hole in the 3D model on the un-patterned illumination images.

Described herein are methods and apparatuses (e.g., systems, devices, etc., including intraoral scanners) that may detect and/or refine interproximal spaces in 3D digital dental models by correlating the results of the 3D mesh analysis (based on the use of structured light to generate a point cloud and therefore the 3D digital model) with one or more non-structured light images (e.g., color images, including white-light images). Non-structured light images may be used to generate 3D information that may be used for refinement of the 3D dental model mesh in order to improve accuracy of the interproximal spaces in particular. In general, the methods and apparatuses described herein may do this by increasing the accuracy of the position(s) of the oner or more cameras used to take the non-structured light images. In particular, these methods and apparatuses may correct the positional information of the camera(s) of the intraoral scanner relative to the 3D model (and/or relative to the structured light images or digital models, e.g., point cloud, derived therefrom). Thus, the methods and apparatuses described herein may use one or more techniques to correct the camera position(s) for the non-structured light images (and therefore the positions of the non-structured light image relative to the surface of the 3D digital model) so that the non-structured light image(s) may be aligned with the 3D digital model and used to correct or modify the 3D digital model, including correcting irregularities (e.g., filling in gaps/holes, etc.) in the 3D digital model.

In some examples, the methods and apparatuses described herein may be used with an intraoral scanner that uses sparse structured light to capture a 3D surface, e.g., of the subject's dentition. As mentioned, although the use of sparse structured light may be relatively low cost and quick, it may result in a lower resolution, particularly in the interproximal regions. The methods and apparatuses described herein may overcome these limitations by using the non-structured light (e.g., in some examples, white light) images that are typically taken by the same scanner interleaved with the structured light imaging, but at a high resolution. These higher resolution 2D non-structured light images may be used to enhance the 3D surface modeling, and in particular, enhancing the sparse point cloud derived from the structured light images, resulting in a more reliable and correct 3D surface model, and therefore more accurate dental/orthodontic treatment and appliances.

For example, described herein are methods that may include: identifying an interproximal region, which may include a region having a surface irregularity (e.g., hole, gap or region suspected to have a surface hole) in a 3D model that is derived from a plurality of structured light images taken from an intraoral scan of a patient's teeth; identifying a set of non-structured light illumination images taken during the intraoral scan of the patient's teeth (e.g., each of which including a region of the patient's teeth corresponding to the interproximal region including the actual or suspected surface hole in the 3D model); modifying (e.g., correcting or refining) a camera position of a camera corresponding to each of the non-structured light illumination images of the set, which in some cases may include finding an alignment transform that is derived from a comparison of features in a features map of the one 3D model relative to the camera with corresponding features from the non-structured light illumination image; and correcting the surface hole in the 3D model, e.g., using one or more points generated from the modified camera positions and the non-structured light illumination images in combination with points from a point cloud derived from the structured light images from which the 3D model was derived. In general, the methods and apparatuses described herein may refer to identifying and correcting holes or gaps in the 3D surface model in the interproximal region of the dentition, however these methods and apparatuses may apply to detection and correction of any irregularity, including gaps, holes, bumps, discontinuities, and lower-resolution regions. Any of the examples and description provided herein with respect to holes may be applied to any surface irregularity.

The methods described herein may be methods of modifying (e.g., enhancing or improving, including increasing the accuracy and/or resolution of) a 3D digital model of a subject's dentition. For example, a method may include: identifying an interproximal region including a surface hole in a 3D model derived from a plurality of structured light images from an intraoral scan of a patient's teeth; identifying a set of non-structured white-light illumination images taken during the intraoral scan of the patient's teeth including a region of the patient's teeth corresponding to the interproximal region including the surface hole in the 3D model; removing non-structured white-light illumination images from the set in which a camera angle between a camera taking the non-structured white-light illumination image and the region corresponding to the interproximal region including the surface hole is greater than a threshold and/or wherein the camera separated from the surface of the region corresponding to the interproximal region by a distance that is greater than a distance threshold value; modifying a camera position of a camera corresponding to each of the non-structured white-light illumination images of the set with an alignment transform derived from a comparison of features in a features map of the one 3D model, wherein the features map corresponds to a depth map or a height map, relative to the camera with corresponding features from the non-structured white-light illumination image; correcting the surface hole in the 3D model using one or more points generated from the modified camera positions and the non-structured white-light illumination images in combination with points from a point cloud derived from the structured light images from which the 3D model was derived, wherein using the one or more points generated from the modified camera positions and the non-structured white-light illumination images in combination with points from a point cloud comprises using a radial basis function.

The non-structured light illumination images may comprise one or more of: white light (WL) images, near infrared (near-IR) images, and/or fluorescent images. The non-structured light image may uniformly illuminate the area of the image; note that the uniform illumination may vary in intensity of the image and the term uniform illumination is used in contrast with structured light images, in which the pattern of the light applied results in a nonuniform illumination of the image.

Any of these methods may also include selecting the set of non-structured light images so that only images that include the interproximal region having the hole (or suspected to include the holes) are included and analyzed in later steps. For example, any of these methods may include removing any non-structured light illumination images from the set of non-structured light illumination images. This selection may include removing any non-structured light (e.g., WL) images from the set of images in which the camera angle between the camera taking the non-structured light illumination image and the region corresponding to the interproximal region including the actual or suspected surface hole is greater than a threshold (e.g., about 5 degrees, about 7 degrees, about 10 degrees, about 12 degrees, about 15 degrees, about 18 degrees, about 20 degrees, about 22 degrees, about 25 degrees, about 27 degrees, about 30 degrees, about 32 degrees, about 35 degrees, about 37 degrees, about 40 degrees, about 42 degrees, about 45 degrees, about 50 degrees, about 55 degrees, about 60 degrees, about 65 degrees, about 70 degrees, about 75 degrees, etc.) and/or wherein the camera is spaced apart from the surface of the region corresponding to the interproximal region by a distance that is greater than a distance threshold value (e.g., about 1 mm, 2 mm, 3 mm, 4 mm, 5 mm, 6 mm, 7 mm, 8 mm, 9 mm, 10 mm, 12 mm, 15 mm, 17 mm, 18 mm, 19 mm, 20 mm, 21 mm, 22 mm, 23 mm, 24 mm, 25 mm, 30 mm, 35 mm, 40 mm, etc.).

Any appropriate features map may be used. For example, the features map may correspond to a depth map. In some example, the features may correspond to a height map.

In any of these methods, using the one or more points generated from the modified camera positions and the non-structured light illumination images in combination with points from a point cloud may comprise using a radial basis function. Any appropriate radial basis function may be used.

In general, these methods may include correction of the surface of the 3D model, including but not limited to correcting holes and/or irregularities in the surface of the 3D model, using one or more points generated from the modified camera positions and the non-structured light illumination images. In some examples this may include identifying the one or more points from rays projected from the modified camera position to the region of the patient's teeth corresponding to the interproximal region including the surface hole in the 3D model on the non-structured light illumination images.

Any of these methods may include confirming, from the set of non-structured light illumination images, that the surface hole comprises a gap.

Also described herein are systems configured to perform any of these methods. These systems may include or be part of an intraoral scanner. In some cases these systems may be separated from, but used with an intraoral scanner. For example the system may be configured to communicate with an intraoral scanner. In some examples the system may be integrated into an intraoral scanner. For example, a system may include: an intraoral scanner comprising one or more cameras; one or more processors; and a memory storing a set of instructions, that, when executed by the one or more processors, cause the one or more processors to perform a method comprising: identifying an interproximal region including a surface hole in a 3D model derived from a plurality of structured light images from an intraoral scan of a patient's teeth; identifying a set of non-structured light illumination images taken during the intraoral scan of the patient's teeth including a region of the patient's teeth corresponding to the interproximal region including the surface hole in the 3D model; modifying a camera position of a camera corresponding to each of the non-structured light illumination images of the set with an alignment transform derived from a comparison of features in a features map of the one 3D model relative to the camera with corresponding features from the non-structured light illumination image; and correcting the surface hole in the 3D model using one or more points generated from the modified camera positions and the non-structured light illumination images in combination with points from a point cloud derived from the structured light images from which the 3D model was derived. The systems described herein may be configured to perform the method after scanning or while scanning.

For example, a system may include: an intraoral scanner comprising one or more cameras; one or more processors; and a memory storing a set of instructions, that, when executed by the one or more processors, cause the one or more processors to perform a method comprising: identifying an interproximal region including a surface hole in a 3D model derived from a plurality of structured light images from an intraoral scan of a patient's teeth; identifying a set of non-structured white-light illumination images taken during the intraoral scan of the patient's teeth including a region of the patient's teeth corresponding to the interproximal region including the surface hole in the 3D model; removing non-structured white-light illumination images from the set in which a camera angle between a camera taking the non-structured white-light illumination image and the region corresponding to the interproximal region including the surface hole is greater than a threshold and/or wherein the camera separated from the surface of the region corresponding to the interproximal region by a distance that is greater than a distance threshold value; modifying a camera position of a camera corresponding to each of the non-structured white-light illumination images of the set with an alignment transform derived from a comparison of features in a features map of the one 3D model, wherein the features map may correspond to a depth map or a height map, relative to the camera with corresponding features from the non-structured white-light illumination image; correcting the surface hole in the 3D model using one or more points generated from the modified camera positions and the non-structured white-light illumination images in combination with points from a point cloud derived from the structured light images from which the 3D model was derived, wherein using the one or more points generated from the modified camera positions and the non-structured white-light illumination images in combination with points from a point cloud comprises using a radial basis function.

Also described herein are computer-readable storage medium comprising instructions which, when executed by a computer, cause the computer to carry out any of the methods described herein.

As mentioned above, the apparatuses and methods described herein may use one or more non-structured light images to correct errors, irregularities and/or fill in gaps in a 3D digital model, particularly in interproximal regions. The ability to make such corrections may depend at least in part on the ability to align the 2D images with the 3D digital model. Thus any of these methods and apparatuses may include software, hardware and/or firmware to align the 2D images with the 3D model with an extremely high degree of accuracy. This alignment may be based on the accurate determination (or correction) of the position of the camera relative to the surface of the dentition in the 3D model. Since the cameras are rigidly coupled to the hand-held scanning tool (e.g., wand), identifying a correction or more accurate position of one camera provides the accurate correction in position of all of the cameras. The alignment of the 2D image may include generating a transform for the 2D image and/or the camera(s).

Although multi-modal intraoral scanners may stitch together images taken between structured light images by interpolating the position of the camera taking the image between the structured light images, this interpolation alone may not be sufficient to improve the accuracy of the position. Accurately interpolating the position of the camera(s) in relation to the teeth (e.g., the 3D model of the teeth) presents challenges due to the complexities involved, either due to computational demands or time constraints. Even in cases in which interpolation is improved by the use of sensors, such as inertial measurement sensors (IMUs) on the scanning tool (e.g., wand) to accommodate wand movement, these methods still include some margin of error. The methods and apparatuses described herein may use feature matching (e.g., such as, but not limited to edge matching), employing a map (e.g., depth map, height map, etc.) that is obtained from the 3D point cloud and/or 3D model, to determine a precise camera positioning. This method may include aligning features derived from a non-structured light illumination image (such as a white light, near-IR, or fluorescent light image) that was captured during the process of taking structured light scans (before, between, or after these scans). It essentially matches features extracted from the non-structured light image with features present in the point cloud (like a dense point cloud) and/or a mesh for which accurate camera data is available. Further, these techniques may be streamlined so that they may be performed very rapidly and may use a minimum number of processing resources, such as by utilizing specific feature subsets. In some examples, the methods and apparatuses described herein may use edge (and/or shape) detection to compare intraoral scan images taken using different modalities, including comparing a surface rendering modality, such as a structured light modality, with a non-structured light illumination modality, such as a white-light, near-IR, fluorescent, etc. This comparison may provide an alignment transform between the 3D model and the non-structured light illumination image that may allow features from the typically higher resolution, non-structured light illumination image to modify or improve the 3D model. Alternatively or additionally, these methods and apparatuses may determine the position of the scanner relative to the surface (in the time of the uniform image capture); knowing the position of the scanner may allow mapping all of the cameras to the surface.

In some examples, these methods and apparatuses may determine camera position(s) for a non-structured light illumination image of a subject's dentition relative to a 3D model of the subject's dentition. For example, any of the methods and apparatuses described herein may include: identifying edges in a non-structured light illumination image taken from an intraoral scan; determining the location of one or more cameras corresponding to a structured light image taken during the intraoral scan; generating a depth map for the one or more cameras corresponding to the structured light image; identifying edges in the depth map; determining an alignment transform to align edges identified from the non-structured light illumination image with edges identified from the depth map; and modifying a 3D model that is derived from structured light images of intraoral scan using the alignment transform and the non-structured light illumination image.

In general, these methods may be performed while scanning and/or as part of an intraoral scan. Alternatively, these methods may be performed after the scanning is complete. Thus, any of these apparatuses may be included or be integrated with an intraoral scanner. In some cases all or some of these steps of these methods may be performed locally and/or remotely, including by one or more remote processors.

For example, any of these methods may include taking and/or receiving an intraoral scan of a subject's dentition. The intraoral scan may generally be a scan using an intraoral scanner including one or more cameras that may be on part of a wand or other hand-held (or robotically held) device. The scan may generally include imaging with both patterned illumination (e.g., structured light imaging), and imaging with non-structured light illumination (e.g., white light, near-IR, etc.). The intraoral scanning may include switching between different types of illumination (e.g., different modes of illumination and/or imaging), such as switching imaging between surface imaging using structured light for a brief period (e.g., 200 msec or less, 150 msec or less, 100 msec or less, 75 msec or less, 50 msec or less, 30 msec or less, 25 msec or less, 20 msec or less, 10 msec or less, 5 sec or less, etc.) followed immediately by imaging using one or more additional imaging modes, typically non-structured light illumination modes, such as white light or single-wavelength imaging, fluorescence imaging, etc. Each of these one or more additional imaging modes may be performed for an individual brief period (e.g., less than 500 msec, less than 400 msec, less than 300 msec, etc., less than 200 msec, less than 100 msec, less than 50 msec, etc.). The duration of each imaging mode may be different and may be dynamically adjusted. The method or apparatus may rapidly cycle between two or more different imaging modes, and may collect images corresponding to each mode that may be saved as part of an intraoral data set. The camera(s) may be scanned over the subject's dentition while scanning.

In examples in which the method or apparatus determines an alignment transform by align edges between non-structured light illumination image with edges identified from a depth map based on camera positions relative to a 3D model of the teeth (e.g., from a digital model, point cloud, 3D mesh model, etc.) while performing the intraoral scanning, the method may include the steps of identifying edges in the non-structured light illumination image, determining the location of the one or more cameras, generating the depth map, identifying edges in the depth map, calculating the alignment transform, and modifying the 3D model may be performing while scanning. Alternatively these steps may be performed after the scanning is completed (e.g., as a post processing technique).

These methods and apparatuses may generally use edge mapping (and/or mapping of other features) and in particular may compare and match edges between one or more non-structured light illumination images (e.g., white light images, near-IR images, single wavelength images, fluorescent images, etc.) and a depth map derived from a digital model of the teeth and/or a patterned illumination image, such as a structured light image. For example, a structured light image may generate a digital point cloud of the subject's dentition as the teeth are scanned. Multiple structured light images may result in multiple point clouds that may be stitched together to form a dense point cloud, which may alternatively or additionally be converted into a digital 3D mesh model (e.g., including vertices, edges, and faces that together form a three-dimensional model) of the subject's dentition. The methods an apparatuses described herein may generate a full or partial depth map either directly from the image (e.g., the structured light image), from the point cloud corresponding to the image, from a dense point cloud including/corresponding to the image and/or from the 3D mesh model of the dentition including/corresponding to the image.

Although the methods and apparatuses described herein may identify edges from the non-structured light illumination image(s) and the depth map and use these identified edges to determine the transform the image, and/or the camera positions, in some cases other features may be used, rather than (or in addition to) edges. For example, other features may include shape features, surface features (e.g., fiducial markings, attachments, etc.), or the like. Thus, any of these methods or apparatuses may use one or more of these features in addition to or instead of edges.

In general, these methods and apparatuses may include identifying edges (and/or other features) from the non-structured light illumination image, and in particular, may include identifying a particular subset of edges for comparison with edges based of the depth map. For example, any of these methods and apparatuses may include identifying the edges of the non-structured light illumination, and in particular, identifying a subset of edges that are boundaries between hard, non-moving elements in the dentition (e.g., teeth, screws/anchors, fillings, pontics or any other scan bodies, etc.) and air or soft tissue. For example, these methods and apparatuses may include identifying edges from the non-structured light illumination image comprising one or more of: a tooth-air boundary, a tooth-gum boundary, a tooth/scan-body boundary, a gum/air boundary, a tooth-tooth boundary, and/or a scan-body/air boundary. In some cases tooth/gum or scan-body/gum boundaries may be preferred.

The edges may be labeled with an alphanumeric label, symbol, etc. Thus, any of these methods and apparatuses may include labeling the identified edges as either: a tooth-air boundary, a tooth-tooth boundary, a tooth-gum boundary, and/or a scan-body/air boundary, etc. The type of edge may be used in these methods and apparatuses when comparing the non-structured light illumination image to the depth map.

In general, the identification of the edges may be performed in any appropriate manner. For example, any of these methods may include identifying the edges of the non-structured light illumination image using a trained machine-learning agent to identify the edge of the non-structured light illumination image (e.g., an edge detection machine learning agent). Alternatively or additionally edge detection using image processing techniques such as convolution, filtering, etc. (e.g., Sobel edge detection, Prewitt edge detection, Canny edge detection, Laplacian edge detection, etc.).

The images (e.g., non-structured light illumination images, structured light images, etc.) and/or the 3D models derived from these images may be preprocessed prior or as part of any of these methods. For example, these methods may include preprocessing to crop and/or enhance the image, and/or to remove material (e.g., teeth, lips, etc.) that may be moving while or between scans.

In any of these methods and apparatuses the relative locations of one or more cameras corresponding to the image (e.g., the non-structured light illumination image) may be determined by first determining, setting or presuming a location of one or more cameras relative to the structured light image (and/or the 3D model based on the structured light image). Thus, any of these methods may include determining the location of the one or more cameras corresponding to the structured light image taken during the intraoral scan comprises determining the location the one or more cameras corresponding the structured light image that corresponds to the non-structured light illumination image. In some examples the structured light image that corresponds to the non-structured light illumination image is a structured light image that was taken either immediately before or immediately after (or both before and after) the non-structured light illumination image was taken while scanning. The location of the one or more cameras may be determined relative to a 3D model (e.g., the point cloud, the 3D mesh model, etc.) derived from the structured light image.

In any of these methods and apparatuses, the depth map may be a full or partial depth map. For example, the depth map may generally be generating the depth map comprises generating the depth map from a viewpoint of the one or more cameras. In some cases the depth may be generated just around the subset of edges detected in the non-structured light illumination image (e.g., edges corresponding to and/or labeled as a tooth-air boundary, a tooth-gum boundary, a tooth-tooth boundary, and/or a scan-body/air boundary, etc.).

Identifying edges in the depth map may include identifying a sub-set of edges corresponding to the edges identified from the non-structured light illumination image. The method may include calculating the alignment transform by calculating the alignment transform in six spatial degrees of freedom (e.g., x, y, and/or z translation, rotation about x, y and/or z).

Any of these methods and apparatuses may include creating the alignment transform by identifying points in the depth map corresponding to the edges identified from the non-structured light illumination image. As mentioned, any of these methods and apparatuses may include using a subset of the edges identified from the non-structured light illumination image that correspond to a tooth-air boundary, tooth-gum boundary, tooth-tooth boundary, and/or a scan-body/air boundary in six degrees of freedom to minimize the difference in the sum of the squares of a distance between corresponding points of the edges. Creating the alignment transform may include iteratively checking alternative transforms in six degrees of freedom to minimize the difference between the edges (e.g., using a sum of the squares of a distance between corresponding points of the edges, or any other appropriate technique). The alternative transforms may correspond to putative positions of the camera for the non-structured light illumination image.

Alternatively or additionally, in any of these methods and apparatuses, calculating the alignment transform may include using a trained machine-learning agent (e.g., an edge matching machine learning agent) to align edges identified from the non-structured light illumination image with edges identified from the depth map. The edge detection trained machine learning agent may be the same as or different than the edge matching machine-learning agent. Any of the trained machine learning agents described herein may be trained pattern-matching agents, and may generally be an artificial intelligence agent. The machine learning agent may be a deep learning agent. In some examples, the trained machine learning agent (matching agent) may be trained neural network. Any appropriate type of neural network may be used, including generative neural networks. The neural network may be one or more of: perceptron, feed forward neural network, multilayer perceptron, convolutional neural network, radial basis functional neural network, recurrent neural network, long short-term memory (LSTM), sequence to sequence model, modular neural network, etc. The trained machine learning agent may be trained using a training data set comprising labeled alignment transforms, and images taken from intraoral scans (e.g., non-structured light illumination images of dentition, depth maps derived from dentition, etc.). In any of these examples, the trained machine-learning agent may determine a label for an edge and the correct position of the edge. The alignment (e.g., the results of the transform) may be performed using a “geometrical” and/or iterative technique.

In any of these methods and apparatuses the method may include iteratively repeating the steps of generating the depth map, identifying edges and calculating the alignment transform, and using a corrected camera position for the one or more cameras, until a maximum number of iterations has been met or until a change in the corrected camera position is equal to or less than a threshold.

In some examples the method may be directed to identifying the alignment transform that may allow precise comparison between the non-structured light illumination image and the 3D model of the subject's dentition and/or a structured light image or 3D model based on the structured light image. In some cases the methods and/or apparatuses may apply the alignment transform to modify the 3D model or the one or more images on which the 3d model is based. For example, the methods and apparatuses described herein may modify the 3D model using the alignment transform and the non-structured light illumination image comprises correcting a surface of the 3D model. This modification may include correcting the surface of the 3D model (e.g., to add or remove points, vertices, edges, faces, etc.). In some cases the method or apparatus may be used to correct specific regions, including in particular crowded regions, such as the regions between teeth (e.g., interproximal regions, etc.), where the resolution of 3D models based on structured light may be lower than with non-structured light illumination images. The alignment transform may be used to allow direct comparison between one or more region of the dentition by identifying correspondence between the high-resolution non-structured light illumination image and the 3D model. Thus, gaps, holes, opening, etc. within the 3D model may be corrected or adjusted based on the non-structured light illumination image.

Any of these methods may include displaying, storing and/or transferring the modified 3D model.

Also described herein are apparatuses (e.g., devices and systems, including software and/or firmware) for performing any of these methods. These systems may include one or more processors and memory storing instructions (e.g., a program) for performing the method using the processor. A processor may include hardware that runs the computer program code. The term ‘processor’ may include a controller and may encompass not only computers having different architectures such as single/multi-processor architectures and sequential (Von Neumann)/parallel architectures but also specialized circuits such as field-programmable gate arrays (FPGA), application specific circuits (ASIC), signal processing devices and other devices.

In any of these apparatuses the system may be part of or may include an intraoral scanner. For example, described herein are systems comprising: an intraoral scanner comprising one or more cameras; one or more processors; and a memory storing a set of instructions, that, when executed by the one or more processors, cause the one or more processors to perform a method comprising: identifying edges in a non-structured light illumination image taken from an intraoral scan; determining the location of the one or more cameras corresponding to a structured light image taken during the intraoral scan; generating a depth map for the one or more cameras corresponding to the structured light image; identifying edges in the depth map; determining an alignment transform to align edges identified from the non-structured light illumination image with edges identified from the depth map; and modifying a 3D model that is derived from structured light images of intraoral scan using the alignment transform and the non-structured light illumination image.

Alternatively, the apparatuses described herein may be configured to operate separately from the intraoral scanner, either locally or remotely (e.g., on a remote server) to which intraoral scan data is transmitted.

All of the methods and apparatuses described herein, in any combination, are herein contemplated and can be used to achieve the benefits as described herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A better understanding of the features and advantages of the methods and apparatuses described herein will be obtained by reference to the following detailed description that sets forth illustrative embodiments, and the accompanying drawings of which:

FIG. 1 schematically illustrates one example of a method of improving and/or refining a 3D digital model of a patient's dentition within interproximal regions.

FIG. 2A illustrates one example of an intraoral scanner that may be adapted for use as described herein.

FIG. 2B schematically illustrates an example of an intraoral scanner configured to generate a model of subject's teeth using any of the methods described herein.

FIGS. 3A-3E illustrate an example of a method of improving and/or refining interproximal regions of a 3D digital model of a patient's dentition taken by an intraoral scanner as described herein. FIG. 3A shows an input point cloud derived from structured light (SL) images taken by an intraoral scanner. FIG. 3B is an example of a surface reconstruction formed from the point cloud, showing an interproximal region including a surface hole. FIG. 3C illustrates an image of the 3D model surface in which the surface hole has been filled. FIG. 3D shows the modified 3D point cloud in which new additional points have been added to fill the surface hole. FIG. 3E shows a reconstruction of the surface derived from the new points and the original point cloud points.

FIG. 4 schematically illustrates an example of a portion of a method including identifying irregularities (e.g., holes) in a 3D digital model of a patient's teeth.

FIG. 5 schematically illustrates an example of a portion of a method including selecting and reviewing images (e.g., non-structured light illumination images) that include regions corresponding to the identified irregularities (e.g., holes) in the 3D digital model of the patient's teeth.

FIGS. 6A-6D illustrate examples of non-structured light illumination images that include a region corresponding to the identified holes in the interproximal regions of a 3D digital model of the patient's teeth.

FIGS. 7A-7D show examples of transformation of non-structured light illumination images, in which the camera positions for the camera(s) corresponding to the non-structured light illumination images have been corrected as described herein.

FIG. 8 schematically illustrates an example of using the aligned non-structured light illumination images to correct/fill holes in the interproximal region of a 3D digital model using rays projected from the corrected camera position and the non-structured light illumination images as well as the 3D digital model.

FIG. 9A illustrates an example of a non-structured light illumination image in which the corrected camera position (corresponding to the non-structured light illumination image) may be used to generate rays and additional points for correcting/filling holes in the 3D digital model.

FIG. 9B shows an example of a plurality of rays derived from non-structured light illumination images such as that shown in FIG. 9B may be used to generate points to correct/fill in the sparse interproximal regions.

FIGS. 10A-10B schematically illustrates an example of a method for adding points to a point cloud (and ultimately the model surface) corresponding to a 3D digital model of a patient's teeth. FIG. 10A shows a first portion of the method. FIG. 10B shows a second portion of the method for correcting/filling a surface of a 3D digital model of a patient's teeth.

FIGS. 11A and 11B illustrating correction of the surface of an interproximal region of a 3D model of a patient's dentition using the methods described herein. FIG. 11A shows the uncorrected interproximal surface, and FIG. 10 B shows the corrected interproximal surface, which has been corrected as illustrated in FIG. 1.

FIG. 12 schematically illustrates one example of a technique for correcting the alignment of a camera of a non-structured light illumination image that may be used as part of the methods described herein.

FIGS. 13A-13B illustrate one example of edge detection of a non-structured light illumination image that may be part of the technique shown in FIG. 12.

FIGS. 14A-14D illustrate an example of the technique including aligning a non-structured light illumination image with a 3D model of a subject's teeth similar to that shown in FIG. 12. FIG. 14A is an example of a non-structured light illumination image (e.g., white light image), showing the edge detection within the image. FIG. 14B shows an example of a depth map derived from a structured light image showing edges marked. FIG. 14C is a comparison between the edge of the non-structured light illumination image and the edges from the depth map of FIG. 14B. FIG. 14D shows the comparison after determining the alignment transform and aligning the non-structured light illumination image accordingly.

FIG. 15 schematically illustrates one example of a technique for correcting the alignment of a camera of a non-structured light illumination image that may be used as part of the methods described herein.

### DETAILED DESCRIPTION

Intraoral scanners may provide detailed, three-dimensional (3D) models of a subject's dentition. Described herein are methods and apparatuses that may improve the 3D model, and in particular may improve the interproximal regions of a 3D digital model of the subject's dentition.

These methods and apparatuses may generally include identifying one or more regions of the 3D digital model, and in some cases interproximal regions, including one or more surface irregularities such as gaps or holes in the surface. The 3D digital model in these methods and apparatuses may be formed from an intraoral scanner that uses structured light images scanned by a scanning tool (e.g., wand) having one or more cameras. For example, in some cases the method or apparatus may identify an interproximal region including a surface hole in the 3D model using a module (e.g., algorithm, trained machine learning agent, etc.) that identifies the hole(s) in the 3D model. In some cases the module may identify the irregularities by examining the point cloud corresponding to the surface in order to identify regions having a density of points that is below a threshold density. The threshold density may be set, or may be adjustable, including user-adjustable.

Once the irregularities (e.g., holes) have been identified, the method/apparatus may identify a set of non-structured light illumination images taken during the intraoral scan of the patient's teeth including a region of the patient's teeth corresponding to the interproximal region including the surface hole in the 3D model. The non-structured light illumination images may be collected (or in some cases, marked). This set of non-structured light illumination images may be refined, by selecting only those images that fit a selection criterion so that only non-structured light illumination images that will be useful in refining the surface of the 3D model may be included. In some cases the number of non-structured light illumination images in the set may be reduced to a maximum number, which may further help simplify and speed up the process.

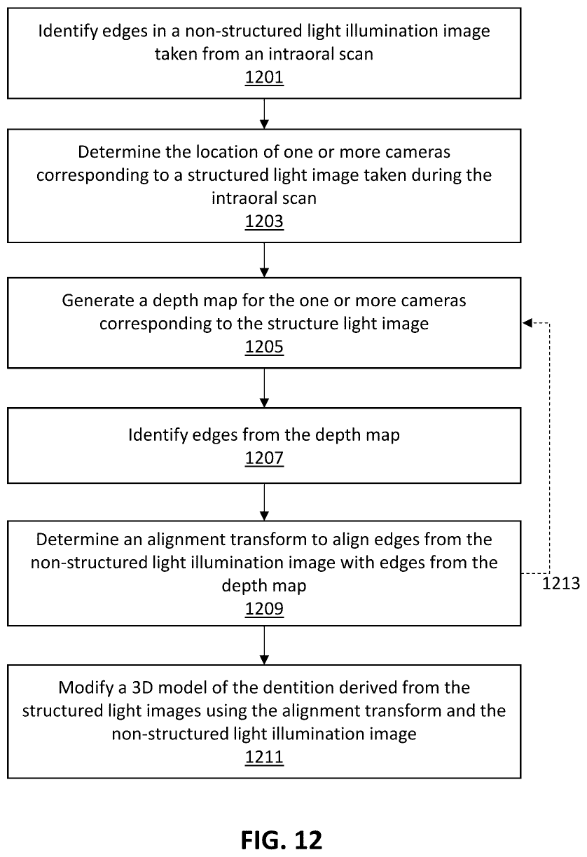

These methods and apparatuses may then determine an accurate camera position for each of the non-structured light illumination images in the set. The camera positions may be corrected (e.g., modified) by modifying a camera position of a camera corresponding to each of the non-structured light illumination images of the set with an alignment transform derived from a comparison of features in a features map of the one 3D model relative to the camera with corresponding features from the non-structured light illumination image. Any appropriate technique for correcting the camera position (typically using a mapping based on the 3D surface and identifying corresponding features between the mapping and the 2D image) may be used; an example of such techniques are described below in reference to FIGS. 12-15.

The irregularities within the interproximal region(s), e.g., holes, may then be corrected using the corrected camera position and the corresponding non-structured light illumination 2D image. In the case of holes or gaps, the holes/gaps may be filled; in the case of other irregularities, the surface may be revised. For example, a hole in the 3D digital model may be filled in or corrected using one or more points generated from the modified camera positions and the non-structured light illumination 2D images in combination with points of the point cloud forming the 3D digital model; the point cloud may be derived from the structured light images.

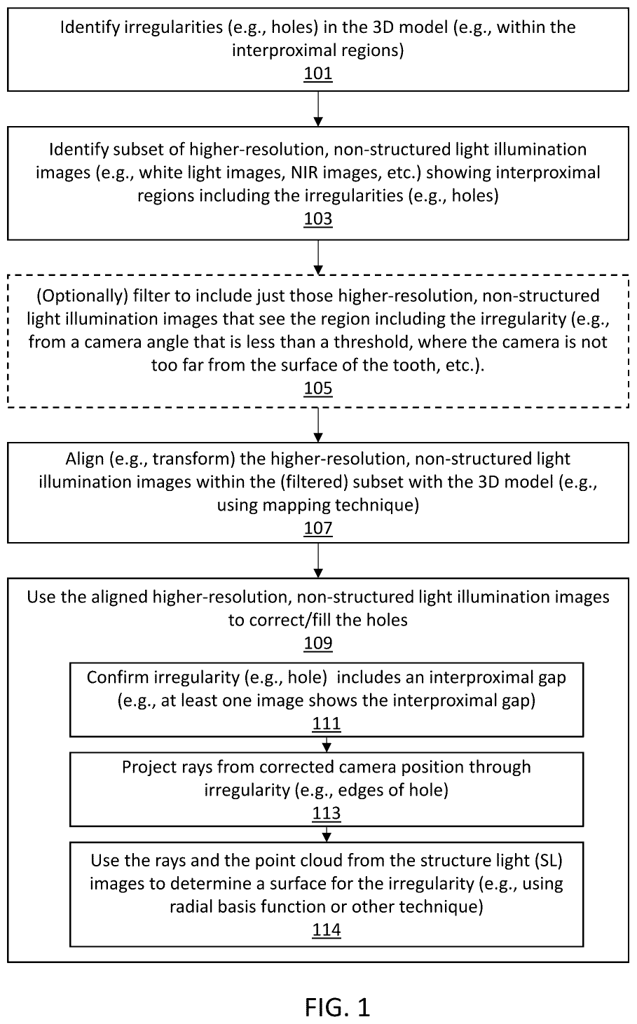

For example, FIG. 1 illustrates one example of a method for improving, repairing or revising the interproximal regions of a 3D digital model. In this example, the method includes identifying irregularities, such as (but not limited to) holes or gaps in the 3D digital model within the interproximal region 101. This step may be performed manually or, preferably, automatically. In some cases, this step may be performed by a module that identifies irregularities of the 3D digital model within the interproximal region.

Once the irregularities have been identified, a set of 2D non-structured light illumination images that include this region may be identified. In general, the 2D non-structured light image may come from the same intraoral scan that was used to generate the 3D digital model 103. In particular, the 2D non-structured light illumination images may be taken concurrently with the structured light images used to generate the 3D digital model. The 2D non-structured light illumination images may be taken between each structured light image. In general, the 2D non-structured light illumination images may be taken at a slightly higher resolution than the structured light images. As mentioned, the 2D non-structured light illumination images may be taken with one or more wavelengths such as white light (WL), near-infrared (NIR), fluorescent light, etc. Thus, a subset of higher-resolution, non-structured light illumination images showing interproximal regions including the irregularities (e.g., holes) may form a set of images (e.g., a set of 2D non-structured light illumination images).

Optionally, the set of non-structured light illumination images may be filtered to include just those non-structured light illumination images that show (e.g., “see”) the region including the irregularity with sufficient detail 105. For example, the method may include removing images (or not including them when forming the set of images) that have a camera angle that is larger than a threshold camera angle value, such as greater than about 5 degrees (e.g., greater than about 7 degrees, about 10 degrees, about 12 degrees, about 15 degrees, about 18 degrees, about 20 degrees, about 22 degrees, about 25 degrees, about 27 degrees, about 30 degrees, about 32 degrees, about 35 degrees, about 37 degrees, about 40 degrees, about 42 degrees, about 45 degrees, about 50 degrees, about 55 degrees, about 60 degrees, about 65 degrees, about 70 degrees, about 75 degrees, etc.). Alternatively, this may be expressed as including images in which the angle between the camera and the region of the surface of the 3D model including the irregularity is less than a maximum threshold camera angle (less than about 75 degrees, less than about 70 degrees, less than about 65 degrees, less than about 60 degrees, less than about 55 degrees, less than about 50 degrees, less than about 45 degrees, less than about 42 degrees, less than about 37 degrees, less than about 35 degrees, less than about 32 degrees, less than about 30 degrees, less than about 27 degrees, less than about 25 degrees, less than about 22 degrees, less than about 20 degrees, less than about 18 degrees, less than about 15 degrees, etc.).

The set of non-structured light illumination images may be filtered to include just those images in which the camera taking the 2D image is within a threshold distance from the surface of the teeth/gingiva, e.g., include those images in which the camera length is not too far from the surface of the tooth. The set of images may be filtered based on other criteria, including quality of the non-structured light illumination images (e.g., resolution, etc.).

Once the set of non-structured light illumination images is complete, the method may include determining a correction of the camera position for each of the non-structured light illumination images in the set. This step may include or may be referred to as aligning the non-structured light illumination images with the 3D model 107, and/or developing a transform to align the non-structured light illumination images with the 3D model, and/or developing a transform including correcting the camera position relative to the surface of the teeth (e.g., the surface of the 3D model generated by the structured light images). Note that the alignment of the non-structured light illumination images may be equivalent to the corrected camera positions, since once the camera position is corrected the non-structured light illumination images may be readily aligned.

The non-structured light illumination images in the set of non-structured light illumination images may then be used with the corrected camera positions (or, equivalently, the aligned non-structured light illumination images) to correct the 3D model surface, e.g., filling in the holes/gaps 109. In some example, this step may include confirming that the irregularity (e.g., hole) includes an interproximal gap (e.g., at least one image shows the interproximal gap) 111 or a type of irregularity that may be corrected. In some cases the type of irregularity, such as the hole, may modify the step of repairing the irregularity. For example, interproximal irregularities between teeth in a face-to-face configuration and/or back-to-back configuration may be repaired differently than other holes, including holes that are within the gingival region rather than teeth.

The repair may include projecting rays from the camera taking the image using the corrected camera position, so that the ray travels to or through the identified irregularity (e.g., through the edges of the irregularity, e.g., hole) 113. The projected rays may then be used to identify one or more points that should be on the surface of the correct tooth surface. For example, the method may use the rays and the point cloud from the structure light (SL) images to determine a surface for the irregularity (e.g., using radial basis function or other technique) 114.

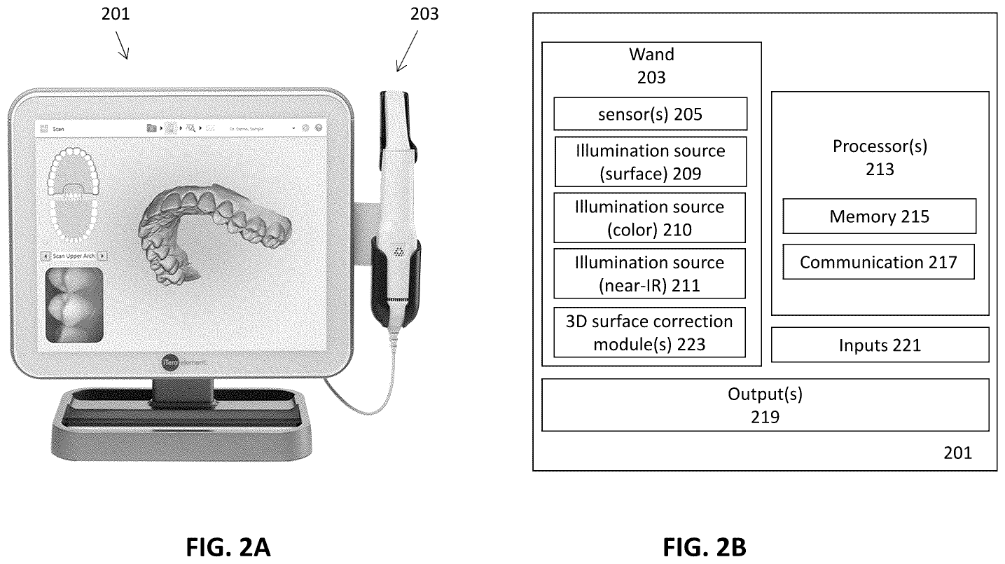

FIGS. 2A-2B illustrate one example of a system that may perform the methods described herein. In some example, the system may include or be integrated into (e.g., part of) an intraoral scanner. The intraoral scanner may be configured to generate digital 3D model of the subject's dentition. The system 201 may include a scanning tool, shown as a wand 203 in this example. As shown schematically in FIG. 2B, an exemplary system including an intraoral scanner may include a wand 203 that can be hand-held by an operator (e.g., dentist, dental hygienist, technician, etc.) and moved over a subject's tooth or teeth to scan. The wand may include one or more sensors 205 (e.g., cameras such as CMOS, CCDs, detectors, etc.) and one or more light sources 209, 210, 211. In FIG. 2B, three light sources are shown: a first light source 209 configured to emit light in a first spectral range for detection of surface features (e.g., visible light, monochromatic visible light, etc.; this light does not have to be visible light), a second color light source (e.g., white light between 400-700 nm, e.g., approximately 400-600 nm), and a third light source 111 configured to emit light in a second spectral range for detection of internal features within the tooth (e.g., by trans-illumination, small-angle penetration imaging, laser florescence, etc., which may generically be referred to as penetration imaging, e.g., in the near-IR). Although separate illumination sources are shown in FIG. 2B, in some variations a selectable light source may be used. The light source may be any appropriate light source, including LED, fiber optic, etc. The wand 203 may include one or more controls (buttons, switching, dials, touchscreens, etc.) to aid in control (e.g., turning the wand on/of, etc.); alternatively or additionally, one or more controls, not shown, may be present on other parts of the intraoral scanner, such as a foot petal, keyboard, console, touchscreen, etc.

The light source may be matched to the mode being detected. For example, any of these apparatuses may include a visible light source or other (including non-visible) light source for surface detection (e.g., at or around 680 nm, or other appropriate wavelengths). A color light source, typically a visible light source (e.g., “white light” source of light) for color imaging may also be included. In addition a penetrating light source for penetration imaging (e.g., infrared, such as specifically near infrared light source) may be included as well.

The apparatus 201 may also include one or more processors, including linked processors or remote processors, for controlling the wand 203 operation, including coordinating the scanning and in reviewing and processing the scanning and generation of the 3D model of the dentition, which may include correcting/revising the 3D model using the 2D images as described herein. As shown in FIG. 2B the one or more processors 213 may include or may be coupled with a memory 215 for storing scanned data (surface data, internal feature data, etc.). Communications circuitry 217, including wireless or wired communications circuitry may also be included for communicating with components of the system (including the wand) or external components, including external processors. For example the system may be configured to send and receive scans or 3D models. One or more additional outputs 219 may also be included for outputting or presenting information, including display screens, printers, etc. As mentioned, inputs 221 (buttons, touchscreens, etc.) may be included and the apparatus may allow or request user input for controlling scanning and other operations. The apparatus may also include communication circuitry for controlling communication with one or more external processors. An output (e.g., screen, display, etc.) may be provided.

The system may include one or more modules 223 (hardware, software and/or firmware) for performing the methods described herein, including identifying irregularities, aligning the camera positions and/or 2D non-structured light images, and fixing the irregularities (e.g., filling the gap(s)), as described herein.

The intraoral scanners providing the scan image and/or 3D model of the dentition may be configured to operate by interleaving and cycling between surface-model generation scans (e.g., structured light images) and non-structured light illumination images (e.g., white light images, near-IR images, etc.). For example, an intraoral scanner may alternate between surface scanning by structured light scanning and one or more other scanning modalities (e.g., internal feature scanning, such as penetration imaging scanning using florescence and/or near-IR scanning). After positioning the scanner adjacent to the target intraoral structure to be modeled, the wand may be moved over the target while the apparatus automatically scans the target. As part of this method, the system may alternate (switch) between scanning a portion of the target (e.g., tooth) using a first modality (e.g., surface scanning, using structured light emitted in an appropriate wavelength of range of wavelengths) to collect surface data such as 3D surface model data, and scanning with one or more second modalities, e.g., white light (e.g., view finding), fluorescent, near-IR light, etc. After an appropriate duration in the first modality, the apparatus may switch to a second modality (e.g., white light) for a second duration to collect one or more images. The apparatus may then switch to one or more additional imaging modalities. Each of these imaging modalities may be referred to as a frame, N, and may generally scan approximately the same region of the target, as the speed of scanning and switching between these modes (e.g., the duration, dn, and separation, tn, may each be relatively fast. At the time of the switch, the coordinate system between the two modalities is approximately the same and the wand is in approximately the same position, as long as the second duration is appropriately short (e.g., 200 msec or less, 150 msec or less, 100 msec or less, 75 msec or less, 50 msec or less, 30 msec or less, 25 msec or less, 20 msec or less, 10 msec or less, 5 sec or less, etc.). Alternatively or additionally, the method and apparatus may extrapolate the position of the wand relative to the surface, based on the surface data information collected immediately before and after collecting the internal data. Thus, as described above, the apparatus may interpolate an initial estimated position for the wand, and therefore the cameras, for each of the non-structured light illumination images. This interpolation may roughly account for the small but potentially significant movement of the wand during scanning, and the use of the edge-detection methods described herein may correct for the movement.

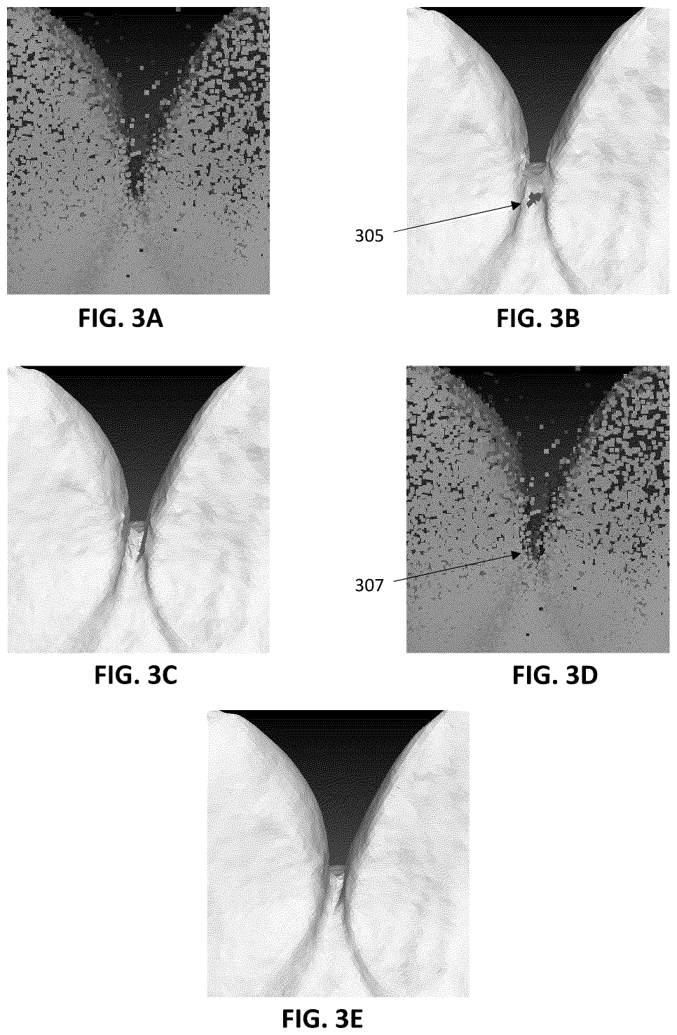

FIGS. 3A-3E illustrate one example of a method, similar to that shown in FIG. 1, above, of improving the surface of an interproximal region of a 3D model of a subject's dentition. FIG. 3A shows an image of the initial point cloud including an interproximal region. This point cloud may be derived from structured light images taken using an intraoral scanner as described herein. A 3D surface may be reconstructed from the point cloud, but may include one or more irregularities, as shown in FIG. 3B. For example, the dense point cloud shown in FIG. 3A may then be transformed into a mesh, such as a triangular mesh, digital model. In FIG. 3B, the reconstructed surface has a hole 305 within the interproximal region, as this region does not have a sufficiently high density of points. In general, the hole may be filled by an algorithm using the existing (sparse) points in this region, as shown in FIG. 3C, but this may result in a less accurate rendering of the interproximal region. Since these 3D models may be used for preparing treatment plan for treating the teeth, including making one or more dental appliances, the resulting approximation may be insufficient and may result in problems in treatment and/or fit of dental appliances. The methods and apparatuses described herein may instead provide an alternative that uses 2D images (non-structured light illumination images) to add new points 307 to the surface of the 3D model, as shown in FIG. 3D. After adding the new points, the surface may be constructed/reconstructed (e.g., using a Poisson technique), as shown in FIG. 3E, eliminating the hole and resulting in a much more accurate and higher resolution 3D digital model.

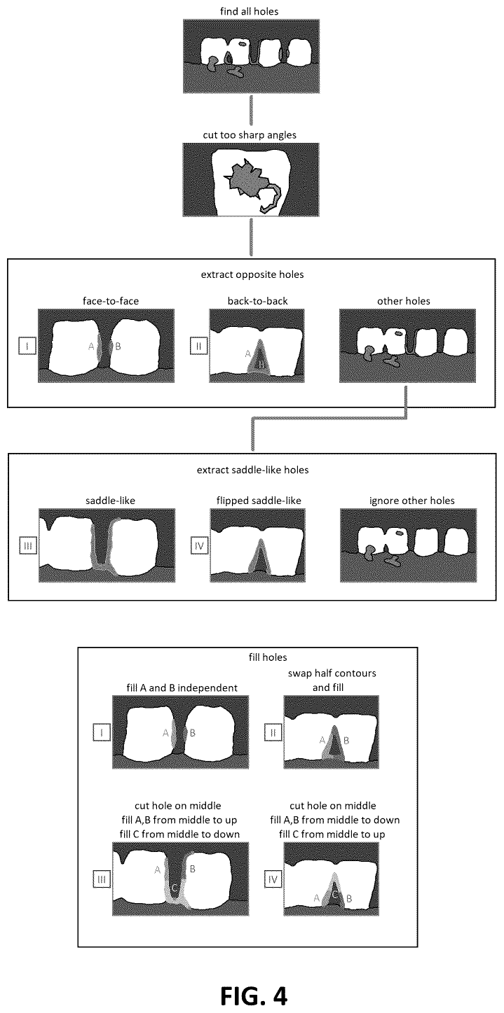

In practice any of these methods and apparatuses may include identifying irregularities, such as holes or gaps, in the 3D model. FIG. 4 schematically illustrates on example of a method of identifying and/or characterizing the irregularities. In FIG. 4, the method may include all holes in the 3D surface. This may be done algorithmically, e.g., by scanning the surface for discontinuities, and/or too-sharp angles (having a change in curvature greater than a threshold valve). Alternatively, the irregularities may be identified using machine learning, e.g., using a trained machine learning agent.

The irregularities may be divided up into different types that may be detected separately and/or using different techniques. For example, irregularities on opposite sides of the interproximal space, such as face-to-face or back to back holes, on facing teeth, may be identified; in some cases an irregularity (e.g., hole) on one side of the interproximal space may indicate an irregularity on the opposite tooth. In some cases the irregularity may extend to the gingiva (e.g., in a saddle-like irregularity) and/or may extend to the connection between adjacent teeth (e.g., an inverted or flipped saddle-like shape). In some cases irregularities that do not fall into these predetermined categories may be ignored or repaired by other techniques. This may include holes in the gingival region.

As mentioned, the category of the irregularity (e.g., hole) that is identified may be used to assist in repairing or modifying the irregularity in the 3D digital model. For example, face-to-face irregularities may be repaired independently on either side (both teeth and/or one of the teeth) of the interproximal space. Saddle-like irregularities or flipped saddle-like irregularities may be repaired by repairing different regions of the saddle-shaped irregularity differently. For example, in FIG. 4, the irregularities may be divided up into regions that may be repaired differently.

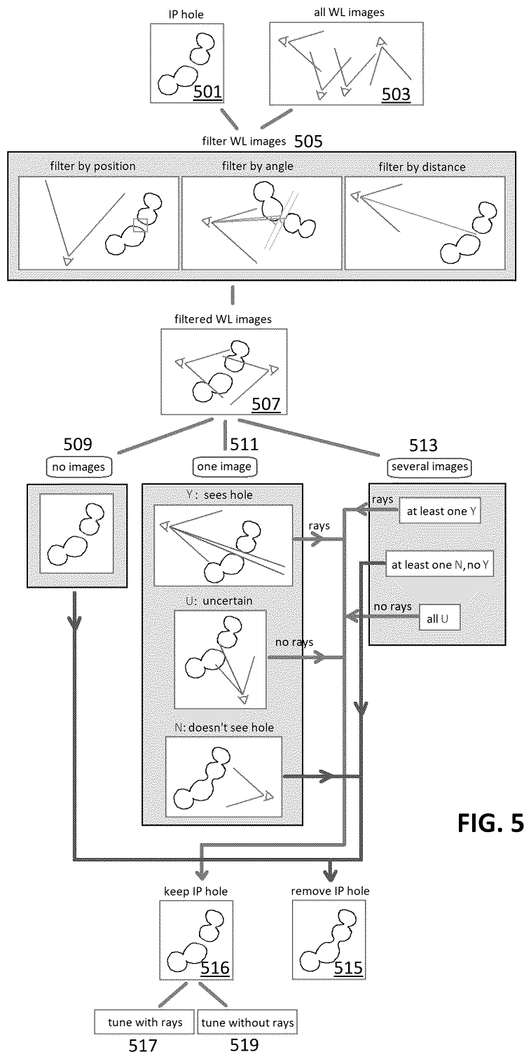

FIG. 5 schematically illustrates a generic method of repairing an irregularity (e.g., hole) within an interproximal region of the teeth. For example, the method may include identifying or receiving a region including an irregularity (e.g., hole) 501, and generating a set of 2D non-structured light illumination images 503 from the same scan that was used to generate the 3D model. As discussed above, the method may optionally include filtering the non-structured light illumination images 505. In This example the non-structured light illumination images are white-light images, and are filtered by the position of the camera, the angel of the camera relative to the surface of the teeth, and the distance of the camera from the surface of the teeth. The set of non-structured light illumination images may be filtered 507 to remove non-structured light illumination images that are not likely to show the region of the surface of the dentition (e.g., teeth, gingiva, etc.) within the interproximal region that includes the hole from the corresponding region of the 3D digital model.

The set of non-structured light illumination images may then be analyzed to determine how to correct the irregularity based on the number of non-structured light illumination images in the set and/or the images shown in the set. These methods may also use the set of non-structured light illumination images to determine if the irregularity (e.g., hole) corresponds to a separation between the teeth (e.g., the interproximal region) or a gap through the teeth. For example, as shown in FIG. 5, if the identified region of the 3D model having the irregularity does not include any non-structured light illumination images 509, the method may address the irregularity, e.g., filling the hole, by interpolating from the existing points in the point cloud 515, removing the hole in the interproximal region, but with relatively low resolution. Alternatively if the identified region of the 3D model having the irregularity does is shown in only a single non-structured light illumination image, and the non-structured light illumination image in the set shows that the hole is present (e.g., by projecting rays from the camera position to the irregularity such that the rays extend through the interproximal region 511) or result in an uncertain result if the ray does not extend through the irregular region but the angle of the ray relative to the irregularity on the 3D model is greater than a threshold, then the method may include adding additional points to the point cloud forming the 3D model 516 (e.g., from the rays 517 or some other technique 519). However, if the camera is too far from the surface of the teeth or irregularity is not visible from the non-structured light illumination image, then the method may either not correct, or may correct by interpolating from the existing points in the point cloud 515.

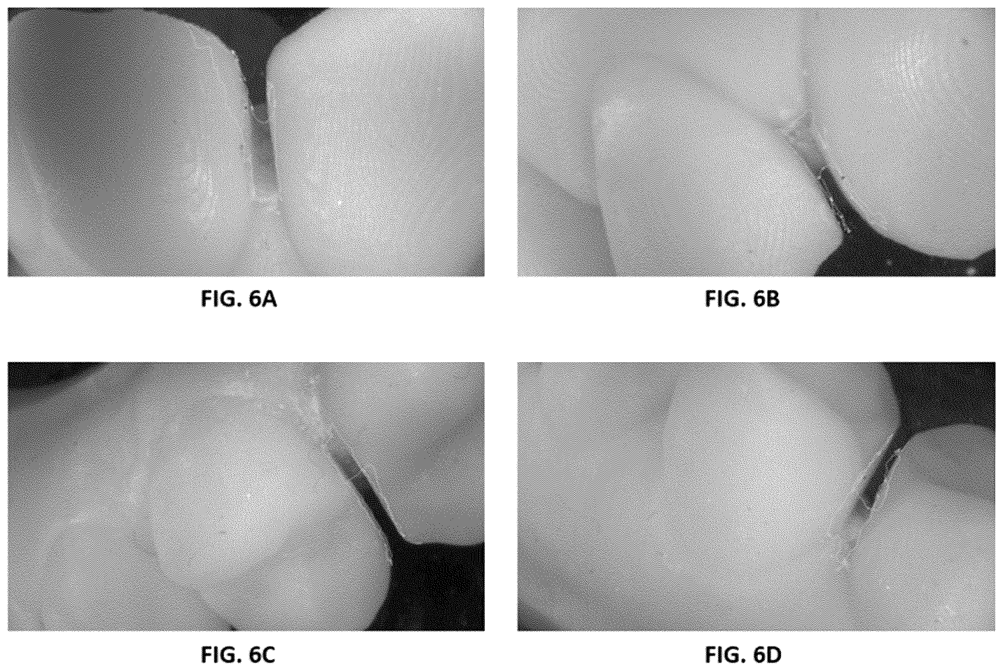

FIGS. 6A-6D illustrate examples of a plurality of different non-structured light illumination images of the same region of the interproximal space having views of an irregular region (e.g., hole) in the interproximal region. In these examples, the same interproximal region is shown from different views, prior to correcting the camera position(s). In FIGS. 6A-6D edge lines are shown on the interproximal regions in each image.

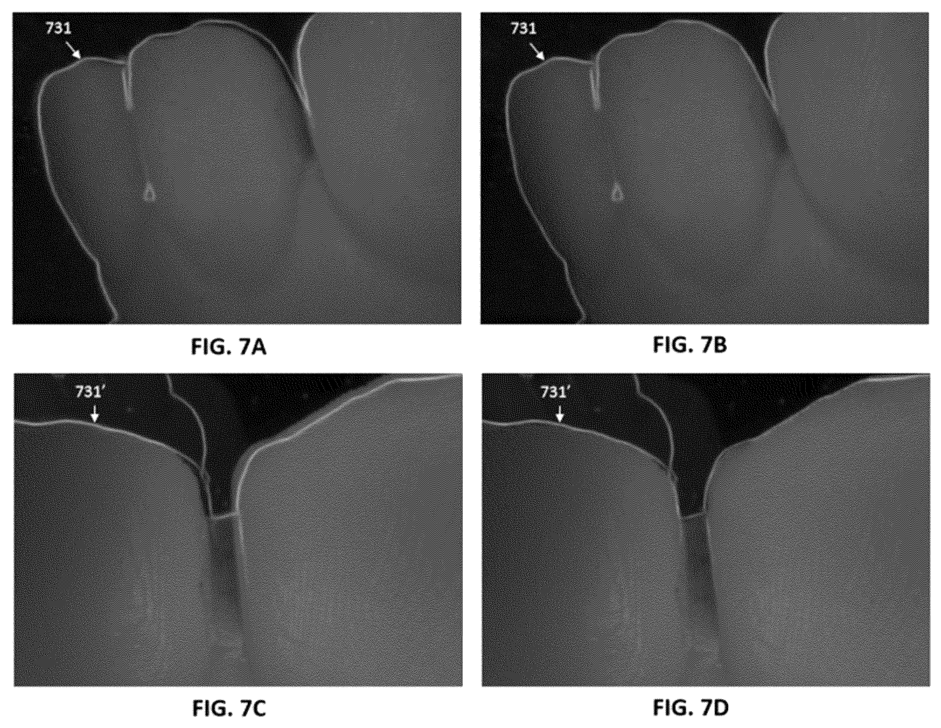

Non-structured light illumination images such as those shown in FIGS. 6A-6D may be aligned by the fine camera-alignment/positioning techniques described below, e.g., in reference to FIGS. 12-15, described in greater detail below. FIGS. 7A-7D illustrate examples of non-structured light illumination images before (FIGS. 7A and 7C) and after (FIGS. 7B and 7D) determining the corrected camera positions and the alignment of the images. FIGS. 7A and 7B illustrate the same non-structured light illumination images before and after transformation once the corrected camera position has been identified. Similarly, FIGS. 7C and 7D show the same non-structured light illumination image before and after correction, respectively. These images illustrate the detection of edge (line 731, 731′) of the non-structured light illumination images. The corrections described herein may be particularly helpful in the interproximal regions. Although in general the intraoral scanner may have a positioning error (between the relative camera taking the non-structured light illumination image and the surface of the teeth) that is relatively small, e.g., less than about 60-70 microns, the average diameter of the interproximal space between the teeth may be only 200 microns or less, which represents a relatively large proportion of the space.

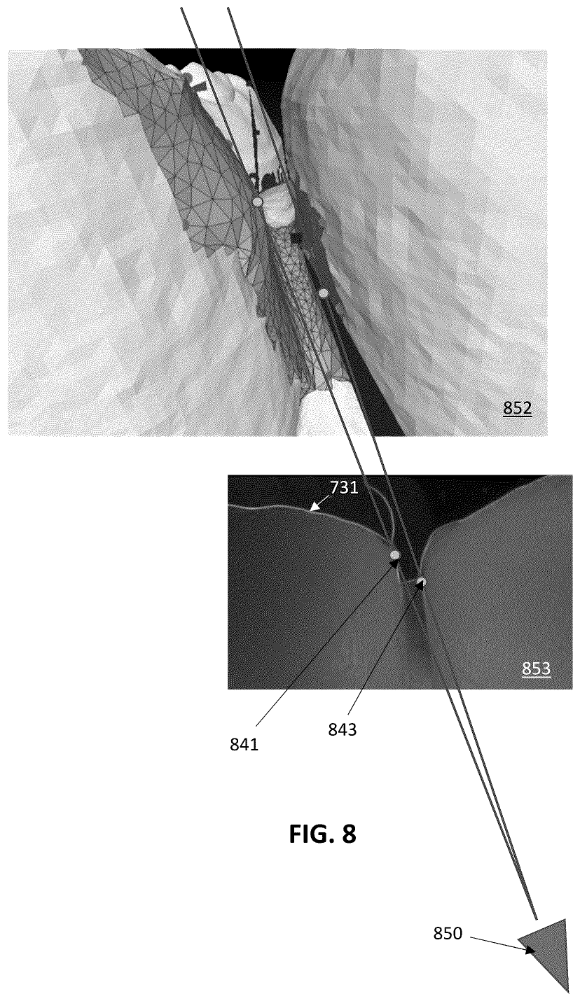

Once the non-structured light illumination image(s) in the set have been aligned, they may be used to repair or modify the irregularity, including filling a gap or hole. For FIG. 8 illustrates one example of a method of repairing an irregular region (e.g., filling a hole) using a non-structured light illumination image and the corrected camera position, and projecting rays from the camera at the corrected position through identified tooth boundary regions 831 of the non-structured light illumination image, and identifying one or more points 841, 843 on the surface of the teeth in the non-structured light illumination image, then translating the identified points to the 3D model using the same relative position of the camera 850.

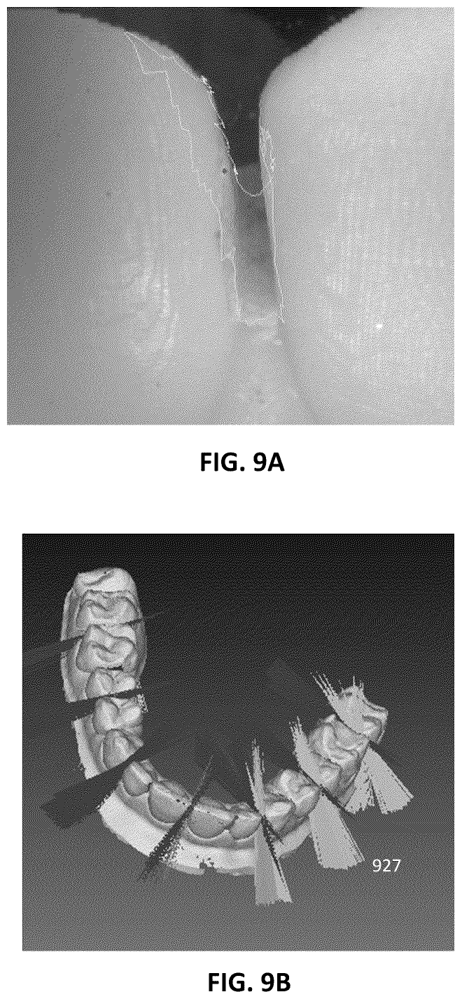

The precision of these computations, when using the aligned non-structured light illumination image and/or camera position, in combination with the 3D model and/or point cloud from the structured light images, may simplify the computation of the surface. This is also illustrated in FIGS. 9A and 9B. In FIG. 9A, a non-structured light illumination image is shown, similar to FIGS. 6A-6B. FIG. 9B illustrates the use of a plurality of rays 927 through the gaps or openings in the interproximal regions of the dentition.

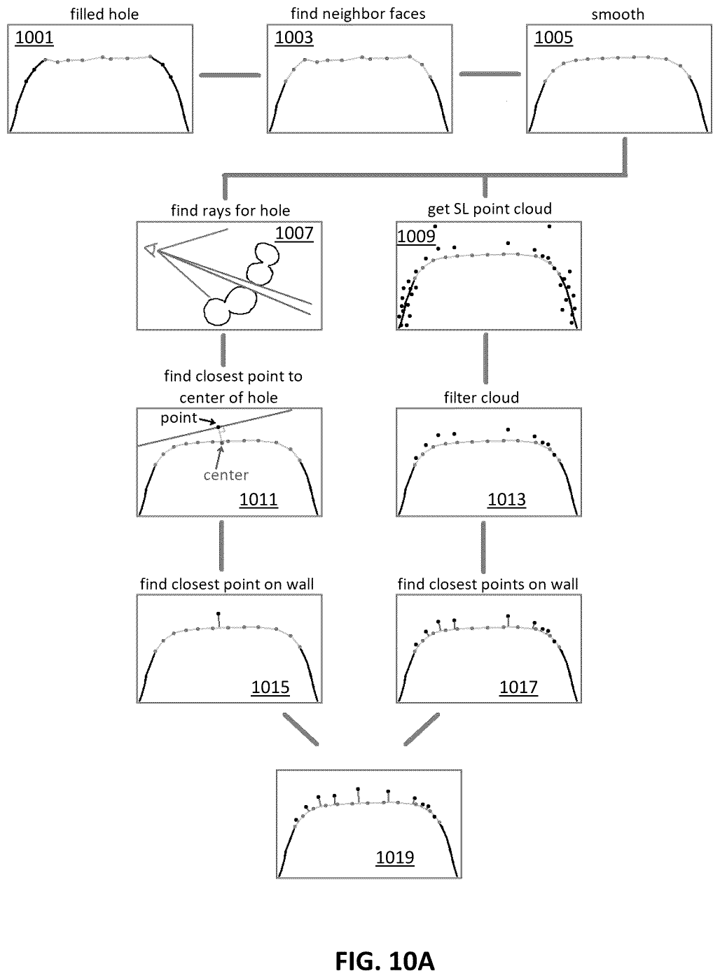

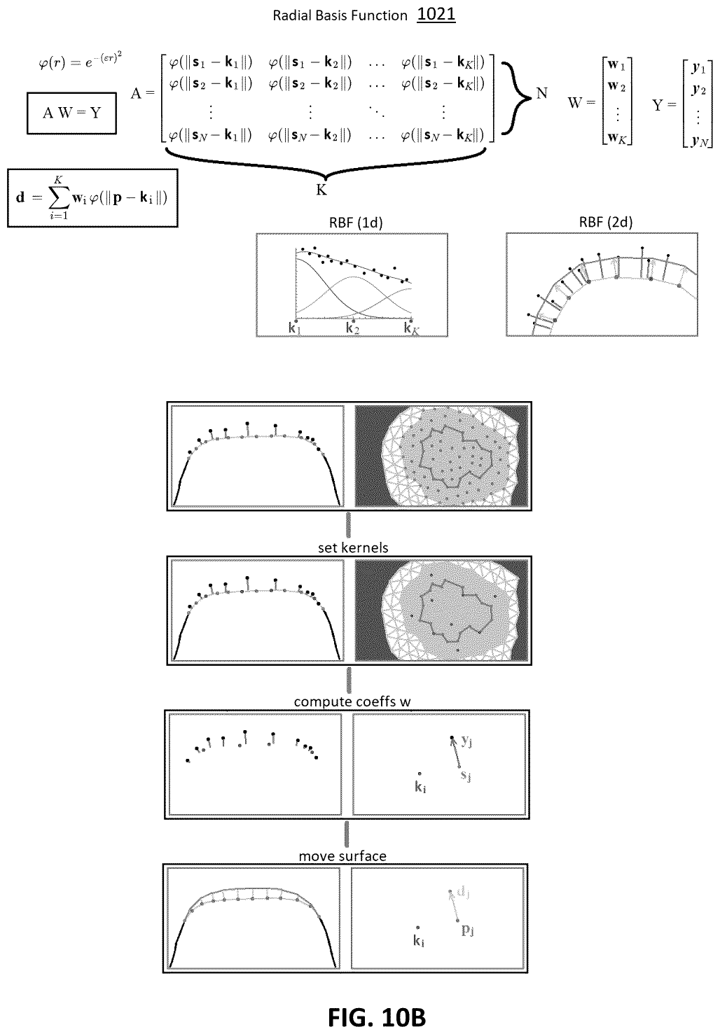

FIGS. 10A-10B illustrate one example of a technique for repairing an irregular region (e.g., filling a hole or gap 1001 in the 3D surface), by adding additional points to the 3D point cloud. In FIG. 10A the 3D model may generally include forming the surface by using the identified points (e.g., in the point cloud from the structured light scan) by identifying points that correspond to neighbor faces 1003 and smoothing the connection between the points 1005. These holes (irregular regions) may be corrected as described above, by identifying non-structured light illumination images, determining the corrected camera positions and/or aligning the images, and using the aligned images to identify rays through and/or around (at the edge regions) of the irregular region/hole 1007. In parallel, the method may include getting the original point cloud that was generated from the structured light image 1009. In some examples, the rays may be used to find points on the ray that are closest to a center of the irregular region (e.g., hole) 1011. For example, these methods may use the rays to identify the closet point on the wall of the 3D model 1015. This additional point may be added to the filtered point cloud 1013 (which may be filtered to remove points that are beyond a maximum distance from the smoothed surface and/or are outside of the irregular region. The points from the original point cloud may also be identified as closets to the smoothed surface 1017. Both the original points from the point cloud (as filtered) and the new points may be combined to form an updated point cloud 1019.

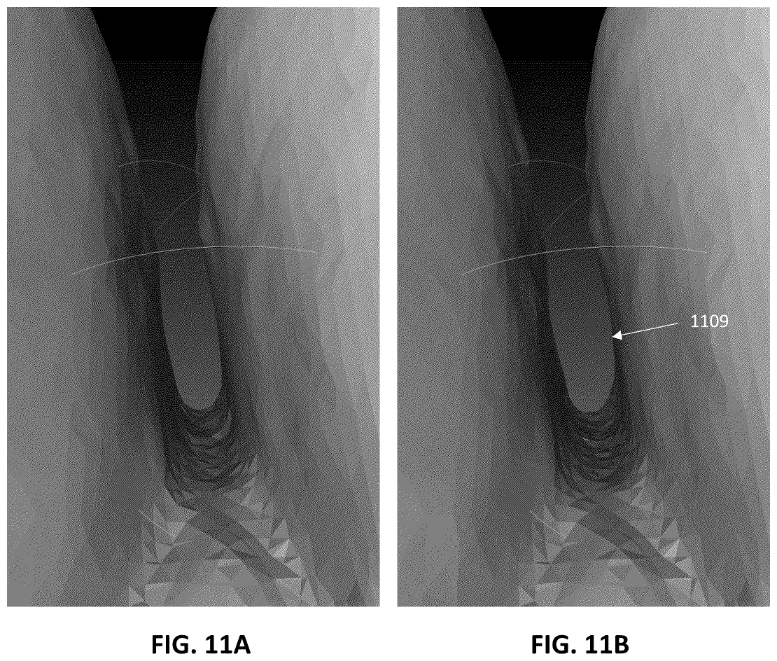

The updated point cloud, in which additional point are added, may be used as input to an algebraic function, such as a radial basis function 1021, in order to generate a new surface, as illustrated in FIG. 10B. The method may include setting kernels for the radial basis function, computing the coefficients and moving the original smooth surface to the new surface based on the results. In general, this may result in an improved and enhanced surface, as illustrated in the example shown in FIGS. 11A and 11B. FIG. 11A shows one example of an uncorrected interproximal region of a 3D digital model of a subject's teeth taken from an intraoral scan using structured light. The methods described above may be used to correct the interproximal region as described above. FIG. 11B shows the revised surface of the interproximal region. In FIG. 11B the interproximal region has expanded slightly 1109, as the surface tracks more closely with the actual tooth surface.

### Camera Position Correction

As mentioned above, any appropriate technique may be used to correct the camera position in the non-structured light illumination images. In more general terms the methods and apparatuses described herein may provide for highly accurate camera position information for images taken while scanning with an intraoral scanner. In particular, these methods and apparatuses may determine the camera position for non-structured light (e.g., uniform) illumination images, such as white light images, near infrared (near-IR) images, fluorescent images, etc. In some cases, the method and apparatuses described herein may provide a high accuracy transform of the position of the one or more cameras (e.g., typically cameras that positionally rigidly coupled) used for capturing the images (e.g., a uniform illuminated image), which may be more accurate than other techniques, such as trajectory interpretation, that attempt to provide the positional information for the camera(s). Because the cameras are rigidly connected relative to each other, such as coupled to a scanning tool (e.g., wand), a single transform may be determined and applied to all of the cameras.

As used herein, non-structured light illumination may refer to images taken with illumination that is not used of generating the 3D model of the teeth, in contrast to structured light images that use patterned light. As mentioned above, the non-structured light may be any appropriate wavelength(s), such as, but not limited to white-light (WL) illumination images, fluorescent images and/or near Infra-RED (NIR) illumination, which gives NIR images. Other types of non-structured light illumination images may include ultraviolet (UV) or any other LED illumination without a mask or pattern. Although the non-structured light illumination (and non-structured light illumination images) described herein may be equivalently referred to as uniform illumination. The image field of view may be uniformly illuminated, although the illumination is not necessarily strictly uniform in intensity across the image field of view. Non-structured light illumination may be used in contrast to structured (e.g. patterned) light. For example, typically illumination using white light and/or near-IR light may change somewhat laterally and in depth, but may change smoothly over the field of view.

An intraoral scanner may take images that may be used to create 3D surface models of the subject's dentition while scanning. For example, structured light images may be taken to generate 3D points while moving the camera(s), generating a 3D point cloud that may be combined, e.g., stitched together, to form the 3D model. The surface of the 3D model may be the meshing of the point cloud. Stitching may be used to estimate the position of the cameras/wand with respect to the surface.

In between capturing the structured light images, the intraoral scanner may also capture uniformly illuminated (e.g., non-structured light illumination) images, such as color images taken with white light, near-IR, etc. The position of the camera when taking these images may be intermediate between the position when taking the structured light images. Thus, the camera position, and in particular, the camera position in space, which may be relative to the resulting 3D model, may be interpolated from the positions of the structured light images taken before and/or after the non-structured light illumination image. Previous attempts to improve the accuracy of this interpolation have used motion sensing, such as an inertial measurement sensor (IMU) to use changes in velocity or rotational acceleration to improve the position estimate, however these techniques still result in an error. These errors may reduce the accuracy of alignment of the non-structured light illumination images with the 3D model, which may be useful both in interpreting the images as well as in improving the 3D model; if the non-structured light illumination images can be accurately aligned with the 3D model (e.g., if the camera position for the non-structured light illumination can be more accurately determined), these images, which may contain information not found in the 3D model, may be used to modify and improve the 3D model. The methods and apparatuses described herein may overcome this error and may improve alignment between the 3D model (and/or images, such as structured light images, on which the 3D model is based) and the non-structured light illumination images taken while scanning.

The methods and apparatuses described herein use edge detection and matching between the non-structured light illumination image(s) and a depth map generated based on a camera position relative to the structured light image (or a 3D model based on the structured light image) to generate a highly accurate alignment transform that can be used to determine an accurate position of the camera(s) for the non-structured light illumination image(s). This may permit the modification of the 3D model based on the non-structured light illumination image(s).

An intraoral scanner may generally include one or more cameras that are rigidly connected in a scanning tool, such as a wand, that may be manually or automatically (e.g., robotically) scanned within the subject's mouth. If there are multiple cameras, the 3D relationship between the cameras may therefore be from calibration of the intraoral scanner. In general the intraoral scanner may interleave scanning of a 3D surface-building scan, such as a structured light capturing scan, and scans of one or more non-structured light illumination images. The 3D surface-building scan such as the structured light scan may be used to generate the digital 3D model of the subject's dentition. For example, each structured light scan capture may create a point cloud, and these point clouds may be stitched together to create a dense point cloud. The dense point cloud may then be transformed into a mesh, such as a triangular mesh, digital model. This process may result in a six degrees of freedom (DOF) transform that also represents the position and angle between the camera(s), e.g., in the wand, and the 3D surface model. Thus, for each of the non-structured light illumination images taken between individual structured light image, the general position of the camera(s) relative to the 6 DOF transformation (e.g., the 3D model of the dentition) may be approximately known by interpolating the camera/wand position from the structured light images taken before and after the non-structured light illumination image. In cases where there are multiple cameras, the cameras may take the images simultaneously, providing multiple, different, viewpoints, corresponding to each of the n cameras. Thus, the position of the scanning tool, e.g., wand, in which the n cameras have a fixed relationship, may be used to determine where all the n cameras were relative to the 3D model based on the 6 degree of freedom transformation.

As mentioned, the multiple different cameras may be rigidly connected relative to each other (e.g., on a wand) so that the relative positions of each camera relative to each other remains fixed. Thus, a single wand position (or a 6 degree position transform) may be true for all of the cameras at the same time. This multi-camera effect is particularly useful since not all cameras will see all (or enough) features of the image. In addition, using a single camera may be more likely to permit some location errors than using multiple cameras. Multiple cameras that provide different angles may therefore result in a much more exact transformation. In addition, some edges cannot be determined for all degrees of freedom. For example, a line may restrict some degrees of freedom but not all; having multi cameras seeing different regions and different edges may therefore solve this problem.

In general the method described herein may use edge detection to determine an alignment transform for the camera position between a non-structured light illumination image and a digital 3D model of the subject's dentition. These methods (and apparatuses for performing them) may include edge detection of the non-structured light illumination images. In some cases only a subset of the edges may be used, which may improve the speed and reduce the processing requirements. For example, the methods described herein may use only a subset of edge that will have corresponding edges in a depth map derived from the structured light image(s), such as edges between the teeth and air, between the teeth and gingiva, between a tooth and the air (e.g., tooth/air boundary), between a tooth and the gingiva (e.g., at tooth-gingiva boundary), between a tooth and a scan-body, between adjacent teeth and/or between regions of a tooth (e.g., grooves, ridges, cusps, etc.), between the gingiva and the air (e.g., a gingiva/air boundary) and/or between a scan body and the air (e.g., a scan-body/air boundary). A scan body may refer to a solid structure within the patient's dentition, such as a screw, post, etc. Note that the types of edges (e.g., tooth/air, gingiva/tooth, gingiva/air, etc.) may behave differently when viewed from different directions. For example, the tooth/gingiva edge may remain in good approximation at same place relative to the object, when viewed from different directions, whereas the tooth/air edge may move relative to the object when viewed from different directions. This phenomena may be considered when iterating in the methods and apparatuses described herein, and may be used to determine if a new estimation of edge position should be computed, or if a height map should be derived and/or modified.

FIG. 12 schematically illustrates one example of a method as described herein. In FIG. 12, the method includes identifying, e.g., computing the edges in all of the images of a frame first frame, N, 1201. These N images may be one or more non-structured light illumination images taken with the one or more cameras. These images may be taken as part of an ongoing intraoral scan of the patient's dentition. Edges may be detected in any appropriate manner, including classical edge detection techniques, such as using convolution, filtering, etc. (e.g., Sobel edge detection, Prewitt edge detection, Canny edge detection, Laplacian edge detection, etc.), and/or using a machine learning agent (e.g., edge identifying machine learning agent). Edges may be characterized, e.g., based on the boundary identified (e.g., tooth/air, scan body/air, gingiva/air, gingiva/tooth, gingiva/scan body, tooth/scan body, etc.). In some cases the edges may be labeled, e.g., the image(s) may be labeled to indicate the edges and edge types. Edges that are not one of these predetermined type may be omitted, including edges within the gingiva, etc.). Edges that result from reflections (e.g., direct reflections) may be false edges, and may be characterized (and may not be used).

The method may further determine the location of one or more (e.g., n) cameras corresponding to a structured light image taken during the intraoral scan 1203. For example, an approximated location of the scanning tool (e.g., wand) may be determined based on the structured light image(s), and/or 3D model derived from the structured light image(s). The estimated position may be based on the structured light image taken at the frames immediately prior to the non-structured light illumination frame, e.g., N−1, and/or immediately after the non-structured light illumination frame, e.g., frame N+1. The method may compute all camera locations relative to the 3D mesh.

A depth map may then be computed for each camera from its viewpoint 1205. For example, a depth map may be generated a for the one or more cameras corresponding to the structure light image based on the computed camera locations determined from the prior or subsequent frame(s). The depth map may include just the subset of edges identified from the illumination. Edges may be identified from the depth map 1207.

The method may then compare the edges detected from the depth map with edges detected from the one non-structured light illumination image(s) to determine an alignment transform 1209. For example, an alignment transform may be calculated to align edges from the non-structured light illumination image with edges from the depth map. This alignment, and the resulting transform, may be done in 3D space, e.g., in six spatial degrees of freedom. The alignment transform may bring the edges from the depth map as close as possible to the edges of the uniform-illuminated image. Any appropriate technique may be used to align the edges. For example, in some cases, this may be done using an iterative closest point algorithm (ICP). Alternatively or additionally, an edge-matching machine learning agent may be used. In some cases the steps of identifying edges from the depth map and matching the edges to the non-structured light illumination image(s) may be combined. For example, the same machine learning agent may be used for both identifying edges in the depth map and matching edge from non-structured light illumination image(s) to the depth map.

The alignment transform may be determined from the edge matching by finding corresponding points for the edges. For example, for each camera, and for each point in the depth map edges, the nearest point from the image edges may be found. The points on the edges from the depth map may correspond to the pixels making up the edge. In some cases, only points that are sufficiently close to one another (e.g., within a threshold distance, such as within 5 pixels, 6 pixels, 7 pixels, 8 pixels, 9 pixels, 10 pixels, 11 pixels, 12 pixels, 13 pixels, 14 pixels, 15 pixels, 16 pixels, 17 pixels, 18 pixels, 19 pixels, 20 pixels, etc.) may be used. If there are multiple close matches, the nearest match may be used. In some cases the methods and apparatuses may limit the edges used to those with similar 2D normal valves. In variations in which the edges are labeled, only points that have the same labels may be used.

Once the corresponding points are identified between the non-structured light illumination image(s) and the depth map, an objective function may be calculated as the sum (or square sum) of distances between the corresponding points from all cameras. The transformation may be optimized by iterating (e.g., as an inner loop) to minimize the objective function. For example, a non-linear optimization may be performed on the 6 degrees of freedom of the wand position to bring the objective function to a minimum. In each step of the minimization, a new transformation may be tested. In some examples the edges of the depth map may be recomputed, while leaving the edges from the non-structured light illumination image(s) intact. For example, the depth map edges may be recomputed using initial x, y, z values of the depth map edges to recompute new x, y, z values after a putative transform is estimate. This putative (e.g., intermediate) transform may be used to project the initial/current x, y, z values onto the cameras with the camera model to determine how well the images match. Thus each putative transform may be tested and modified until the error is sufficiently small or until a limited number of repetitions is reached. In some examples, the putative transform may be considered the ‘best’ transform and may be used to determine a new approximate camera position and this new camera position may be used to repeat steps 1205, 1207 and 1209, e.g., may be used to recompute the depth map 1205, so that edges can again be identified from the depth map 1208 and a new alignment transform may be estimated 1209. This iterative loop may be referred to as the outer loop, shown in FIG. 12 as the dashed line 1213. This outer loop may be optional, but may improve the accuracy of the method. The outer loop may be repeated for a predetermined number of iterations or until a maximum number of iterations have been performed, and/or it may be terminated if the resulting error (distance) between the edges is sufficiently low.

Thus, in general, these methods may initially compute edge positions and labels from the images. As part of the outer loop, the method may compute the depth map and compute edges in 3D. The method may perform the inner loop by projecting the 3D edges using the camera(s) transform and computing the objective function. The transform may be updated to minimize the objective function. The decision to exit the inner and/or outer loop may be made either when a maximum number of iterations (or time) have been reached, or when further improvement is not possible (or is below a threshold).

Once the alignment transformed (e.g., an optimized) has been determined, it may be used to apply the non-structured light illumination image(s) to the 3D model. For example, in some cases the non-structured light illumination image(s) may be used to modify (e.g., correct or adjust) the 3D model of the subject's dentition. Thus, the 3D model of the subject's dentition that is derived from the structured light images may be modified using the alignment transform and the non-structured light illumination image 1211. In some cases the surface of the 3D model may be adjusted, e.g., moving the vertices or points (pixels) in some regions to more accurately reflect the actual tooth position. Gaps or openings in the digital model may be corrected using the coordinating region(s) of the non-structured light illumination image. In some cases the coordinated regions of the non-structured light illumination image may be used to determine boundaries between teeth (e.g. interproximal regions, etc.), and/or may be used to assist in or correct in segmenting the 3D model, e.g., to distinguish tooth, gingiva, etc. In general, the un-patterned illumination image(s) may be used to improve the resolution and detail of the structured light images and/or the 3D model.

### Edge Detection

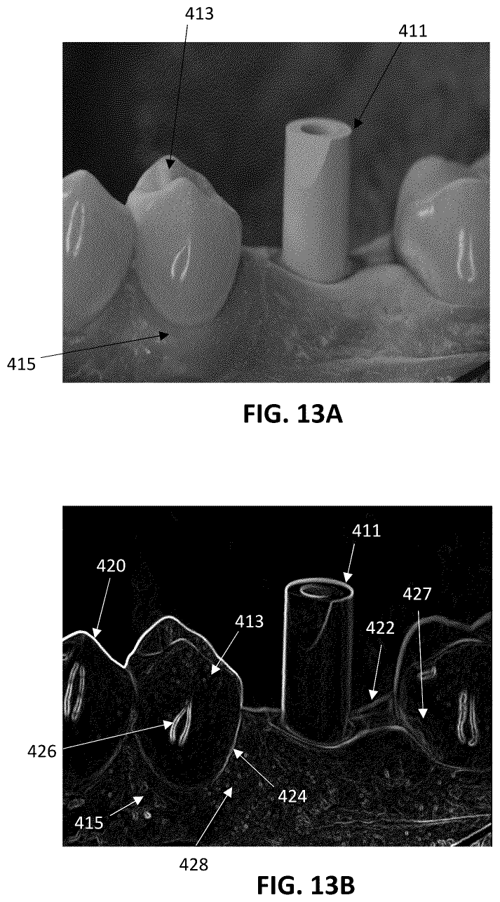

As mentioned above, any appropriate edge detection technique may be included as part of the methods and apparatuses described herein. FIGS. 13A and 13B illustrate one example of edge detection of a non-structured light illumination image. In this example, the non-structured light illumination image includes a tooth 413 and a scan body 411 (a post in this example), extending from the gingiva 415. In general, some of these edges have well-defined depth map counterparts, while others may be less relevant. FIG. 13B shows the non-structured light illumination image of FIG. 13A with edges indicated. In this example some of the less relevant edges include edges from reflections in the image, such as the reflections from the tooth surface 426, edges from texture on the tooth 427, or texture of the gingiva 428, edges from a filling (e.g., edge of the filling, not shown in FIG. 13B), etc. However, more relevant edges may include edges from tooth-air boundary 420, edges from scan body-air boundary 411, edges from the gingiva-air boundary 422, and edges from tooth-gum boundary 424. Other types of edges that may be used may include tooth-tooth edges (boundaries), which may be boundaries between adjacent teeth, and/or may be boundaries within the surface of a tooth, particularly molar teeth, such as cusps, grooves, ridges, and/or fossa on a tooth. In any of these examples, image processing may be performed to help differentiate the different edges types. In some examples a trained machine learning agent may be used. For example, a trained machine learning agent comprising a convolutional neural net may be used. This trained machine learning agent may learn to detect edges directly, or may segment the objects (e.g., teeth, gums, etc.). The segmented image may be used so that edges of the segments are more easily used to detect edges.

The type of edge may be included, e.g., as a label, for the detected edge. This information may be included with the image, and may be used during later steps, including when generating the depth map and/or identifying edges from the depth map. As mentioned, certain types of edge may be preferred, as they may behave differently. For example, edges that are boundaries with air, such as the tooth-air edge, may not refer to a constant object position when viewed from different orientations, while edges between solid objects, such as the boundary between gingiva and a tooth may remain attached to same object location when viewed from slightly different viewpoints.

Creating a Depth Map from a Point of View

A depth map may be formed for the one or more cameras corresponding to the structured light image(s) being analyzed. The depth map may be estimated directly from the structured light image or from the digital model of the teeth corresponding to the structured light image. For example, the depth map may be estimated as the distance, in millimeters and/or pixels from the camera to the surface(s) within the digital model (e.g., the point cloud and/or mesh model).

In some cases, the type (or more specifically, the model) of the camera(s) may be used in this step. In particular, the camera may be known such that for each pixel, it may be known what the ray in space that is traced by the camera. For example, the camera may be modeled as a pinhole or as a pinhole with optical distortion, which may be appropriate for traditional cameras. In some cases the camera may be a non-standard camera model such as a Raxel type of camera, in which each pixel has a direction, but also a starting point which is not the center pinhole. Thus, the depth map may account for the starting point of the corresponding rays from the camera, which may add to the computational load; in some cases, this may be ignored and may still provide sufficient accuracy.

The camera may be positioned in space relative to the 3D model (derived from the structured light image), and may be positioned with six degrees of freedom. The methods and apparatuses described herein may generate a depth map by computing the distance from each pixel, until it hits the object, by going pixel by pixel, finding its corresponding ray, and checking the first time this ray hits the object. This procedure may be performed as is known in the art. A variety of different algorithms are known and may be used for generating a depth map, including both machine-learning based techniques and more classical, non-machine learning techniques.

### Depth Map Edge Detection

In some examples, once the depth map has been generated, it can be treated like an image, and edges may be computed where significant discontinuations are detected. Edge detection may be applied as described above, and any appropriate edge detection may be used. In some cases, for each edge location, the spatial (e.g., x, y, z) position may be recorded. These methods may also identify regions that are continuous in depth, but not continuously normal to the depth map. These normal discontinuities may typically cause a discontinuity of shade in the corresponding uniform illuminated image, and these types of edge may indicate a corner of a scan body, a tooth gum intersection, or the like, but would not typically be found on a tooth surface.

In some examples, another possible way to detect these edges, and specifically to detect tooth-gum boundaries in the depth map, may include using a trained machine learning agent (including, but not limited to, a trained neural net). The same, or a different trained machine learning agent may be used to detect edges from the non-structured light illumination image. As mentioned, the type of edge detected may be stored and/or coordinated with the depth map, which may also be used in comparing or simplifying the comparison and/or alignment of the edges.

The methods described herein (and apparatuses to perform them) may advantageously determine the camera and/or wand position. This allows these methods to estimate depth, translation and rotation (e.g., θx, θx) from a single image, as described above.

In any of these methods, accurate global camera/wand position corresponding to the time of the capture of the non-structured light illumination image may be determined for all or a subset of different cameras using the methods described herein. For example, assuming that there are a plurality of 2D alignment transforms (one per camara, e.g., 6×2D transforms in instances where there are six cameras), the methods described herein may define at least three non-co-linear points on each of the images used (e.g., on each of the 2D uniform field illumination images). The points selected typically include a surface region, i.e. not an air region, in the image; to ensure this, the points may be selected using an output of a segmentation subsystem (e.g., a segmentation modules or a module that segments the 2D image, and that marks the region of the image which are solid, e.g., representing tooth or gingiva). The points may preferably be on a non-flat region of the surface. For each camera there may be three sets of coordinates (e.g., 3×[u, v], where u and v are used to denote the pixel coordinates in the image). The computed image may then be used in combination with the camera transformation to compute adjusted coordinates (e.g., (u′, v′)) for these points, e.g., compute 3×[u′, v′]. These transformed points may then be back projected to the surface (using the depth-map), so we have 3×[u, v]=>3×[x, y, z] for each camera. This procedure may be optimized to find a shared 3D transform that minimizes the error between the all the projected [x′, y′, z′] points of each camera and the transformed 2D points, e.g., [u′, v′]. This minimization can be performed in either 2D or 3D.

In general, the transform may be identified in a variety of different techniques. For example, using a linear technique (e.g., with SVD) a non-linear technique, an iterative, etc. In some cases the depth error may be minimized while back-projecting by using the transformed [u′, v′] and find the inverse 3D transform that will move the projected points to the original positions, e.g., [u, v]. Alternatively, the above technique may be repeated after transforming the 3D surface with the first iteration transform.

Any of the methods and apparatuses described herein may simplify and reduce the computational load of the methods described herein by simplifying the steps of recomputing the depth map from the new viewpoint and/or the iterations (inner loop) used to find the best transform. For example, any of these methods may optionally reduce the number of cameras. After computing the edge map from the WL image, a subset of the cameras corresponding to those including adequate edge regions may be used.

In general, these methods may reduce the depth map computation to reduce and improve the computational load. The initial “guess” of the camera position transform may be initially relatively close, as the camera position(s) may not move substantially between images. Thus the methods described herein may use the edges detected from the non-structured light illumination image to define regions in the depth map (e.g., up to some distance away, such as up to 20 pixels, up to 25 pixels, up to 30 pixels, up to 35 pixels, etc. away) that may be examined to search for an edge in the depth map. Alternatively or additionally, these methods my limit the creation of the depth map to these regions within a predetermined distance from an edge (or subset of edges) identified in the non-structured light illumination images. Thus the depth map may have a different size as compared to the non-structured light illumination image.

Any of these methods may combine the steps of generating the depth map and identifying edges in the depth map, as mentioned above. For example, these methods may include finding an edge or edges in a region of an image (e.g., of a non-structured light illumination image) and creating a depth map around this edge. In some cases if two samples have significantly different values, the method may include searching for a discontinuity between them. These methods may be used with subsets of the cameras and/or sub-sets of the non-structured light illumination images (e.g., down-sampled) images and depth maps. Alternatively, the method described herein may compute the edges in a multi-scale fashion. In some cases the method may start in strongly down sampled image, and may move up in scale. As mentioned above, any of these methods may simplify the camera model used (e.g., model a raxel camera as a pinhole camera).

In some of the methods described here, only stable edges may be used from the detected edges. For example, the depth map may be configured to include only edges that are at consistent places in the object (e.g., between the tooth and gingiva, between scan-body and gingiva, between tooth and scan body, etc.), and may reduce or exclude those edges that may move with the viewpoint (e.g., tooth/air boundaries, etc.). If the methods use only the stable depth map edges, then there may not be a need it iterate (e.g., outer loop iteration) described above. Alternatively, in some examples, the depth map edges may be generated to minimize the need to regenerate or recreate these edges. In some examples, the method may include determining or estimating how the moving depth map edges actually move; for example, by computing the curvature of each edge found, a rough estimate of how this edge moves can be computed. Edges that move less than a threshold may be retained, and edges that move more than a threshold may be rejected.

In any of these examples, the method may include the use of point features, which are not continues edges, which may be identified both in the depth map edges and in the uniform illuminated images. Such point features may be smaller in number than the edges, and can be corresponded with one another be position and characteristics. For example, an edge with a sharp corner could define such a point feature. In some examples the point in which two teeth and the gum meet may be defined as another point feature. Thus, in additional to edges, one or more distinct features, including point features, may be used. Optimizing the alignment transform when using point features may assist in speeding up the process.

As mentioned above, uniform-illumination may refer to white-light (WL) illumination images, fluorescent images and/or near Infra-RED (NIR) illumination, which gives NIR images. Other types of non-structured light illumination images may include ultraviolet (UV) or any other LED illumination without a mask or pattern. Note that the illumination does not have to be strictly uniform in intensity across the image field of view but may be used as a contrast to structured (e.g. patterned) light. For example, typically illumination using white light and/or near-IR light may change somewhat laterally and in depth, but may change smoothly over the field of view.

As mentioned above, these methods and apparatuses may include one or more preprocessing steps, e.g., for removing moving tissue, and/or cropping and/or adjusting the imaging properties (e.g., brightness, contrast, etc.). Moving tissue such as lips, tongue and/or fingers, that may be included in an intraoral scan may be removed from the images (e.g. the intraoral scan images) prior to performing (or in some case while performing) any of these methods. For example, tongue, lips and/or figures may be removed from the 3D surface model, but may exist in the scan, and exists in the images. In some cases, a moving tissue detection network (e.g., a trained machine learning agent) may be used and included to identify and/or remove such objects and/or mark them in the intraoral scan. Thus the methods described above may be performed on the image region used, e.g., without these moving objects and tissues present.

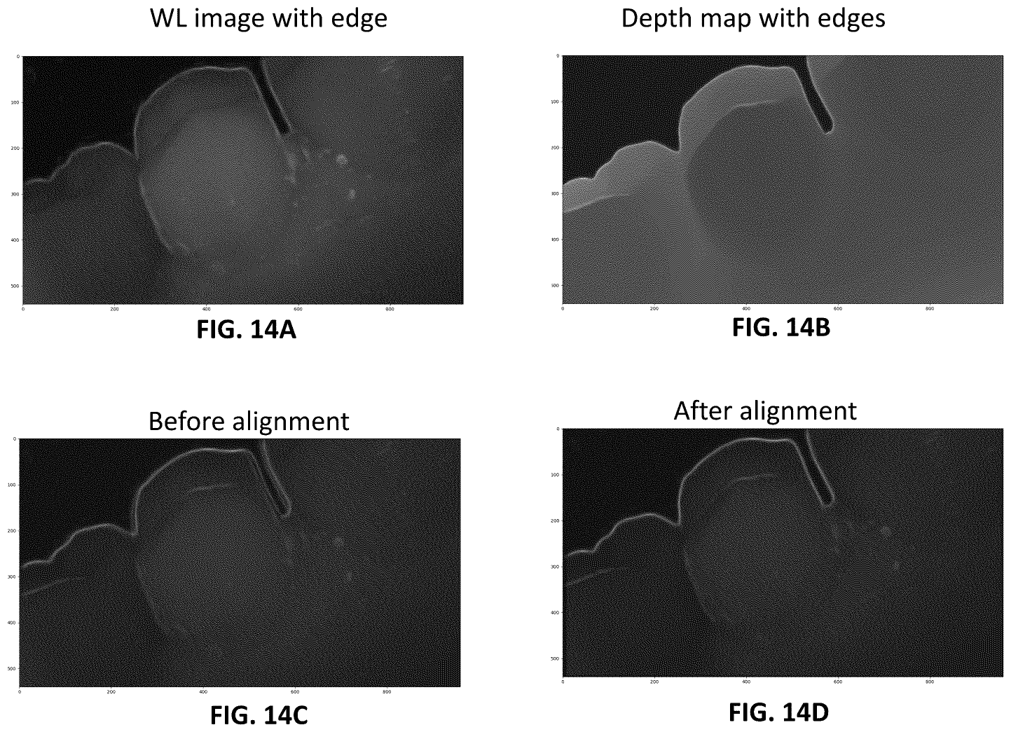

FIGS. 14A-41D illustrate one example of the methods described herein, showing examples of images aligned using this method. FIG. 14A shows an example of an initial non-structured light illumination image (e.g., white-light image) from an intraoral scan. In this example, the image is shown following edge detection as described above. FIG. 14B illustrate an example of a corresponding depth map generated from a 3D model of the dentition that was formed using structured light images taken with the non-structured light illumination image. In FIG. 14B edges have been detected and highlighted in the depth map. FIG. 14C shows an overlay of the edges from the depth map marked on the non-structured light illumination image to show that there is a gap between the edges, prior to alignment. FIG. 14D shows a similar image as FIG. 14C, after alignment has been performed, generating the alignment transform to transform the image so that the edges (also shown in FIG. 14D) nearly perfectly coincide.

### Use of Height Map

A depth map may refer to an image or image channel that contains information relating to the distance of the surfaces of scene objects from a viewpoint; for example, from a camera position, as described above. In some cases, the methods and apparatuses described herein may instead or in addition use a height map (“h-map”). A height map may include surface elevation data. These methods and apparatuses may be similar to those described above, and may be applied to intraoral scan image data that includes both structured light images taken by a scanning tool (e.g., a wand) having a plurality of cameras as the scanning tool is moved over the patient's dentition, as well as a plurality of nonstructured light illumination images taken between the structured light images. These methods and apparatuses (e.g., systems) may also be used to determine highly accurate camera positions for the non-structured light illumination images. This may, in turn, allow the use of information from the non-structured light illumination image(s), including surface details, to be used to modify, correct and/or update the structured light images and/or the 3D model.

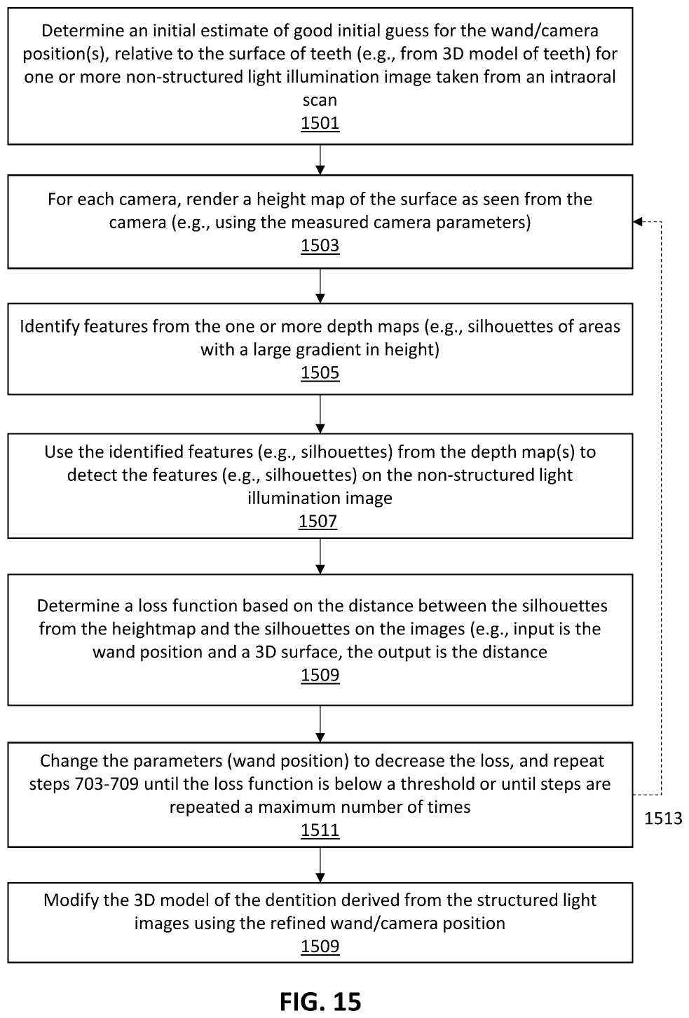

For example, FIG. 15 illustrates any of these methods may include starting with an initial estimate (“guess”) of the position of the camera(s) and/or scanning tool with the camera(s) relative to the surface of the teeth 1501. As described above, this may be estimated based on the approximate position from the structured light scan, which may be modified based on one or more sensors (e.g., IMUs) on the scanning tool. A height map of the surface of the tooth may be generated for each camera of the scanning tool (e.g., wand) from the 3D surface of the tooth relative to each of the cameras 1503.

A height map may be generated from a 3D surface (e.g., the digital 3D model) and the camera (e.g., wand) positions and parameters. The 3D surface and camera positions may be used to calculate the distance from the camera of each of the pixels that the camera sees, e.g. by estimating a ray from the pixel to the surface, the ray following the optical path that we can calculate from the camera parameters and position. Conversely, the methods and apparatuses described herein may perform the opposite technique to determine the height map, e.g., by rendering the surface using the camera parameters and position as the rendering camera. The result of such rendering includes the heightmap (in this context also known as depth map). The renderer can be a standard renderer (e.g. OpenGL), or a differentiable renderer to make the loss differentiable.

Each height map may be generating using measured camera parameters. From each height map, silhouettes may be identified 1505. The silhouettes may be detected as regions having a large gradient in height. These silhouettes may be selected to be a specific range or sub-set o of the possible silhouettes. For example only silhouettes having a minimum gradient (e.g., change) in height may be selected and used in the steps going forward.

Once identified (and/or selected), corresponding silhouettes may be identified in the one or more non-structured light illumination images taken by the intraoral scanner 1507. Because the initial estimates of the camera position(s) are based on the approximate position from the structured light images before and/or after taking the non-structured light illumination image, the silhouettes are likely to be relatively close. The silhouettes identified from the non-structured light illumination image(s) and the silhouettes from the height map may be compared 1509. For example, a loss function may be determined based on the distance between the silhouettes from the height map and the silhouettes on the one or more non-structured light illumination images. The loss function may include all the steps described above (e.g., 1503 to 1507). The input may be the wand/camera position (e.g., parameters) and a surface (e.g., hyperparameter) and the output is the distance.

In any of the methods and apparatuses described herein, the loss function may be made differentiable. For example, rendering the height map of the surface may be performed using differential rendering (e.g., using machine learning techniques, including deep learning techniques, e.g., pytorch3d). Once a differential loss function has been determined, standard optimization techniques (e.g. gradient descent) may be used to optimize the wand/camera position. For examples, these methods may include making the loss function differentiable by rigid body transformation (e.g., x, y, z, theta x, theta y, theta z), in order to make the height map edge differentiable on the rigid body six degrees of freedom.

Using the loss gradient, the parameters (including wand position) may be changed to decrease the loss 1511, and the method may loop back 1513 to the step of rendering the heigh map of the surface, using the new value for the parameters, such as wand position, e.g., step 1503. This process of identifying silhouettes from the depth map, and comparing to silhouettes from the non-structured light illumination images and determining a new loss function may be repeated until either the loss function is less than a threshold valve (which may be another hyperparameter) or until a sufficient amount of time has passed.

Once the loop has been completed, e.g., and the loss function has refined the parameters (e.g., wand/camera position) for a sufficient time and/or until the loss function is less than a threshold valve, these parameters, such as camera position, may be used to modify the 3D model of the patient's dentition, as described above. For example, the revised and refined wand/camera position(s) may be used to align the non-structured light illumination image(s), and the non-structured light illumination image(s) may be used to modify the 3D model, including by filling in missing or erroneous images.

Use with Confocal Images

The concepts embodied as the methods and apparatuses described herein may be used in combination with any volume-generating scan, including but not limited to structured light. For example in some cases confocal images may be taken using an intraoral scanner, which may illuminate using a non-uniform illumination pattern (e.g., checkerboard, etc.) that is not necessarily structured light, but may be used to generate digital surface model information. For example, non-uniformly illuminated white-light image (e.g., confocal image) may bused to generate surface volume (e.g., 3D surface volume) information by an intraoral scanner.

In some examples patterned illumination used to generate a digital surface volume may be a patterned confocal image. For example a patterned illumination system using confocal imaging may provide an imaging of the pattern onto the object being probed and from the object being probed to the camera. The focus plane may be adjusted in such a way that the image of the pattern on the probed object is shifted along the optical axis, preferably in equal steps from one end of the scanning region to the other. The probe light incorporating the pattern may provide a pattern of light and darkness on the object. When the pattern is varied in time for a fixed focus plane then the in-focus regions on the object may display an oscillating pattern of light and darkness. The out-of-focus regions may display smaller or no contrast in the light oscillations. Light incident on the object may be reflected diffusively and/or specularly from the object's surface (however, in some cases the incident light may penetrate the surface and is reflected and/or scattered and/or gives rise to fluorescence and/or phosphorescence in the object). The pattern of the patterned light illumination may be static or time-varying. When a time varying pattern is applied, a single sub-scan can be obtained by collecting a number of 2D images at different positions of the focus plane and at different instances of the pattern. As the focus plane coincides with the scan surface at a single pixel position, the pattern may be projected onto the surface point in-focus and with high contrast, thereby giving rise to a large variation, or amplitude, of the pixel value over time. For each pixel it is thus possible to identify individual settings of the focusing plane for which each pixel will be in focus. By using knowledge of the optical system used, it is possible to transform the contrast information vs. position of the focus plane into 3D surface information, on an individual pixel basis. Thus, in some cases the focus position may be estimated by determining the light oscillation amplitude for each of a plurality of sensor elements for a range of focus planes. For a static pattern, a single sub-scan can be obtained by collecting a number of 2D images at different positions of the focus plane. As the focus plane coincides with the scan surface, the pattern will be projected onto the surface point in-focus and with high contrast. The high contrast gives rise to a large spatial variation of the static pattern on the surface of the object, thereby providing a large variation, or amplitude, of the pixel values over a group of adjacent pixels. For each group of pixels it is thus possible to identify individual settings of the focusing plane for which each group of pixels will be in focus. By using knowledge of the optical system used, it is possible to transform the contrast information vs. position of the focus plane into 3D surface information, on an individual pixel group basis. Thus, the focus position may be calculated by determining the light oscillation amplitude for each of a plurality of groups of the sensor elements for a range of focus planes. A 3D digital model may therefore be used with the confocal patterned light images. For example, a 3D surface structure of the probed object can be determined by finding the plane corresponding to the maximum light oscillation amplitude for each sensor element, or for each group of sensor elements, in the camera's sensor array when recording the light amplitude for a range of different focus planes. The focus plane may be adjusted in equal steps from one end of the scanning region to the other. Preferably the focus plane can be moved in a range large enough to at least coincide with the surface of the object being scanned.

For example, any of these methods may include identifying an interproximal region including a surface hole in a 3D model derived from a plurality of patterned illumination images from an intraoral scan of a patient's teeth. The patterned illumination images may correspond, e.g., to structured light or patterned confocal light images. Any of these methods may then identify a set of un-patterned illumination images (e.g., non-patterned illumination images, such as non-structured light illumination images. The un-patterned illumination images may correspond to uniformly illuminated images, and may be white-light images, etc.). Alternatively in some examples, described below, the un-patterned illumination images may correspond to a portion of a structured light or patterned confocal light image (e.g., excluding the pattern). The un-patterned illumination images may be taken during the intraoral scan of the patient's teeth including a region of the patient's teeth corresponding to the interproximal region including the surface hole in the 3D model.

As discussed above, in cases in which the un-patterned illumination images are taken alternately with the patterned illumination (confocal or structured light) images, any of these methods may include modifying a camera position of a camera corresponding to each of the un-patterned illumination images of the set with an alignment transform derived from a comparison of features in a features map of the one 3D model relative to the camera with corresponding features from the un-patterned illumination images.

Any of these methods may then include correcting the surface hole in the 3D model using one or more points generated from the modified camera positions and the un-patterned illumination images in combination with points from a point cloud derived from the patterned illumination images from which the 3D model was derived.

Thus, in some examples the methods and apparatuses may identify edges in the un-patterned illumination image taken from an intraoral scan and determining a location of one or more cameras corresponding to a patterned illumination image taken during the intraoral scan. These methods may also generate a depth map for the one or more cameras corresponding to the patterned illumination image, identifying edges in the depth map, and may determine an alignment transform to align edges identified from the un-patterned illumination image with edges identified from the depth map. The 3D model that is derived from the patterned illumination images of intraoral scan may be modified using the alignment transform and the un-patterned illumination image, as described above.

### Variations Using Only Patterned Illumination

As mentioned above, in general these methods and apparatuses may improve a 3D model generated by an intraoral scanner, and in particular the interproximal regions, by modifying (correcting, adjusting, filling-in, etc.) one or more regions of a 3D digital model by using additional images, including un-patterned illumination images, taken by the intraoral scanner. In general the intraoral scanner may use patterned illumination (e.g., structured light, patterned confocal imaging, etc.) to generate a 3D digital model of the intraoral cavity (e.g., teeth, gingiva, etc.). The methods and apparatuses described above illustrate methods and apparatuses in which separate 2D images taken with un-patterned illumination may be precisely corrected/aligned (e.g., correcting parameters such as the position of the camera when taking the un-patterned illumination 2D image). However, in some cases it may be possible to use the same patterned images and convert all or a portion of the patterned image(s) into equivalent un-patterned illumination (e.g., uniform illumination) 2D images which may be used to correct the 3D digital model. Because these images are derived from the patterned illumination images the camera parameters (e.g., camera position) do not need to be corrected/aligned, and they may be used directly.

For example, in some cases the method or apparatus may crop or select region from the patterned images that are uniformly illuminated. In variations in which the patterned illumination includes a high-contrast pattern such as a checkerboard, regions of the image may be brightly illuminated. These illuminated regions may be used to modify the 3D digital model. Thus, the methods and apparatuses (e.g., software) may be configured to use only the regions of the pattern that are illuminated above a threshold (e.g., excluding the shaded regions and edges). In some examples the patterned illumination images may be cropped to exclude the pattern (e.g., regions having an illumination intensity that is less than a threshold). In some examples the methods and/or apparatus may modify the patterned illumination image to adjust (make more regular) the overall illumination intensity of the image so that it may be used to modify the 3D digital model in some regions.

The surface hole in the 3D model may be modified (e.g., corrected) using one or more points generated from the un-patterned illumination image(s), which may be white-light illumination images or partial images, in combination with points from a point cloud derived from the patterned illumination images from which the 3D model was derived. Using the one or more points generated from the un-patterned illumination images in combination with points from a point cloud may comprise using a radial basis function as described herein.

Thus, any of the methods and apparatuses described herein may be used with one or more (e.g., a plurality) of un-patterned illumination images that may be taken separately from, e.g., alternating between, the patterned illumination images used to generate the digital 3D model. In some examples the methods and apparatuses described herein may be used with an un-patterned illumination image that is derived from a patterned illumination image (e.g., as a cropped portion, excluding the pattern, a filtered/normalized image, etc.).

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method, the method comprising:
identifying an interproximal region including a surface hole in a 3D model derived from a plurality of structured light images from an intraoral scan of a patient's teeth;
identifying a set of non-structured light illumination images taken during the intraoral scan of the patient's teeth including a region of the patient's teeth corresponding to the interproximal region including the surface hole in the 3D model;
modifying a camera position of a camera corresponding to each of the non-structured light illumination images of the set with an alignment transform derived from a comparison of features in a features map of the one 3D model relative to the camera with corresponding features from the non-structured light illumination image; and
correcting the surface hole in the 3D model using one or more points generated from the modified camera positions and the non-structured light illumination images in combination with points from a point cloud derived from the structured light images from which the 3D model was derived.

2. The method of claim 1, wherein the non-structured light illumination images comprises one or more of: white light images, near infrared (near-IR) images, and/or fluorescent images.

3. The method of claim 1, further comprising removing any non-structured light illumination images from the set of non-structured light illumination images in which a camera angle between a camera taking the non-structured light illumination image and the region corresponding to the interproximal region including the surface hole is greater than a threshold and/or wherein the camera separated from the surface of the region corresponding to the interproximal region by a distance that is greater than a distance threshold value.

4. The method of claim 1, wherein the features map corresponds to a depth map.

5. The method of claim 1, wherein the features map corresponds to a height map.

6. The method of claim 1, wherein using the one or more points generated from the modified camera positions and the non-structured light illumination images in combination with points from a point cloud comprises using a radial basis function.

7. The method of claim 1, wherein correcting the surface hole in the 3D model using one or more points generated from the modified camera positions and the non-structured light illumination images comprises identifying the one or more points from rays projected from the modified camera position to the region of the patient's teeth corresponding to the interproximal region including the surface hole in the 3D model on the non-structured light illumination images.

8. The method of claim 1, further comprising confirming, from the set of non-structured light illumination images that the surface hole comprises a gap.

9. A method, the method comprising:
identifying an interproximal region including a surface hole in a 3D model derived from a plurality of structured light images from an intraoral scan of a patient's teeth;
identifying a set of non-structured white-light illumination images taken during the intraoral scan of the patient's teeth including a region of the patient's teeth corresponding to the interproximal region including the surface hole in the 3D model;
removing non-structured white-light illumination images from the set in which a camera angle between a camera taking the non-structured white-light illumination image and the region corresponding to the interproximal region including the surface hole is greater than a threshold and/or wherein the camera separated from the surface of the region corresponding to the interproximal region by a distance that is greater than a distance threshold value;
modifying a camera position of a camera corresponding to each of the non-structured white-light illumination images of the set with an alignment transform derived from a comparison of features in a features map of the one 3D model relative to the camera with corresponding features from the non-structured white-light illumination image;
correcting the surface hole in the 3D model using one or more points generated from the modified camera positions and the non-structured white-light illumination images in combination with points from a point cloud derived from the structured light images from which the 3D model was derived, wherein using the one or more points generated from the modified camera positions and the non-structured white-light illumination images in combination with points from a point cloud comprises using a radial basis function.

10. A system, the system comprising:
an intraoral scanner comprising one or more cameras;
one or more processors; and
a memory storing a set of instructions, that, when executed by the one or more processors, cause the one or more processors to perform a method comprising:
identifying an interproximal region including a surface hole in a 3D model derived from a plurality of structured light images from an intraoral scan of a patient's teeth;
identifying a set of non-structured light illumination images taken during the intraoral scan of the patient's teeth including a region of the patient's teeth corresponding to the interproximal region including the surface hole in the 3D model;
modifying a camera position of a camera corresponding to each of the non-structured light illumination images of the set with an alignment transform derived from a comparison of features in a features map of the one 3D model relative to the camera with corresponding features from the non-structured light illumination image; and
correcting the surface hole in the 3D model using one or more points generated from the modified camera positions and the non-structured light illumination images in combination with points from a point cloud derived from the structured light images from which the 3D model was derived.

11. The system of claim 10, wherein the non-structured light illumination images comprises one or more of: white light images, near infrared (near-IR) images, and/or fluorescent images.

12. The system of claim 10, wherein the processor is further configured to include the step of removing any non-structured light illumination images from the set of non-structured light illumination images in which a camera angle between a camera taking the non-structured light illumination image and the region corresponding to the interproximal region including the surface hole is greater than a threshold and/or wherein the camera separated from the surface of the region corresponding to the interproximal region by a distance that is greater than a distance threshold value.

13. The system of claim 10, wherein the features map corresponds to a depth map.

14. The system of claim 10, wherein the features map corresponds to a height map.

15. The system of claim 10, wherein using the one or more points generated from the modified camera positions and the non-structured light illumination images in combination with points from a point cloud comprises using a radial basis function.

16. The system of claim 10, wherein correcting the surface hole in the 3D model using one or more points generated from the modified camera positions and the non-structured light illumination images comprises identifying the one or more points from rays projected from the modified camera position to the region of the patient's teeth corresponding to the interproximal region including the surface hole in the 3D model on the non-structured light illumination images.

17. The system of claim 10, further comprising confirming, from the set of non-structured light illumination images that the surface hole comprises a gap.

18. A system, the system comprising:
an intraoral scanner comprising one or more cameras;
one or more processors; and
a memory storing a set of instructions, that, when executed by the one or more processors, cause the one or more processors to perform a method comprising:
identifying an interproximal region including a surface hole in a 3D model derived from a plurality of structured light images from an intraoral scan of a patient's teeth;
identifying a set of non-structured white-light illumination images taken during the intraoral scan of the patient's teeth including a region of the patient's teeth corresponding to the interproximal region including the surface hole in the 3D model;
removing non-structured white-light illumination images from the set in which a camera angle between a camera taking the non-structured white-light illumination image and the region corresponding to the interproximal region including the surface hole is greater than a threshold and/or wherein the camera separated from the surface of the region corresponding to the interproximal region by a distance that is greater than a distance threshold value;
modifying a camera position of a camera corresponding to each of the non-structured white-light illumination images of the set with an alignment transform derived from a comparison of features in a features map of the one 3D model relative to the camera with corresponding features from the non-structured white-light illumination image;
correcting the surface hole in the 3D model using one or more points generated from the modified camera positions and the non-structured white-light illumination images in combination with points from a point cloud derived from the structured light images from which the 3D model was derived, wherein using the one or more points generated from the modified camera positions and the non-structured white-light illumination images in combination with points from a point cloud comprises using a radial basis function.

19. A computer-readable storage medium comprising instructions which, when executed by a computer, cause the computer to carry out the method of: identifying an interproximal region including a surface hole in a 3D model derived from a plurality of structured light images from an intraoral scan of a patient's teeth;
identifying a set of non-structured light illumination images taken during the intraoral scan of the patient's teeth including a region of the patient's teeth corresponding to the interproximal region including the surface hole in the 3D model;
modifying a camera position of a camera corresponding to each of the non-structured light illumination images of the set with an alignment transform derived from a comparison of features in a features map of the one 3D model relative to the camera with corresponding features from the non-structured light illumination image; and
correcting the surface hole in the 3D model using one or more points generated from the modified camera positions and the non-structured light illumination images in combination with points from a point cloud derived from the structured light images from which the 3D model was derived.

20. The computer-readable storage medium of claim 19, wherein the non-structured light illumination images comprises one or more of: white light images, near infrared (near-IR) images, and/or fluorescent images.

21. The computer-readable storage medium of claim 19, further comprising removing any non-structured light illumination images from the set of non-structured light illumination images in which a camera angle between a camera taking the non-structured light illumination image and the region corresponding to the interproximal region including the surface hole is greater than a threshold and/or wherein the camera separated from the surface of the region corresponding to the interproximal region by a distance that is greater than a distance threshold value.

22. The computer-readable storage medium of claim 19, wherein the features map corresponds to a depth map.

23. The computer-readable storage medium of claim 19, wherein the features map corresponds to a height map.

24. The computer-readable storage medium of claim 19, wherein using the one or more points generated from the modified camera positions and the non-structured light illumination images in combination with points from a point cloud comprises using a radial basis function.

25. The computer-readable storage medium of claim 19, wherein correcting the surface hole in the 3D model using one or more points generated from the modified camera positions and the non-structured light illumination images comprises identifying the one or more points from rays projected from the modified camera position to the region of the patient's teeth corresponding to the interproximal region including the surface hole in the 3D model on the non-structured light illumination images.

26. The computer-readable storage medium of claim 19, further comprising confirming, from the set of non-structured light illumination images that the surface hole comprises a gap.
